SEQUENCE LISTING (121 sequences)

SEQ ID NO: 1 - DNA, 20 bp, artificial sequence
  oligonucleotide
agaggagctcagcgtcgact

SEQ ID NO: 2 - DNA, 20 bp, artificial sequence
  oligonucleotide
ggctgaggttgcaactctga

SEQ ID NO: 3 - DNA, 20 bp, artificial sequence
  oligonucleotide
ccaggcaggagcaactcctt

SEQ ID NO: 4 - DNA, 20 bp, artificial sequence
  oligonucleotide
ttgaatagcacattggttgg

SEQ ID NO: 5 - DNA, 20 bp, artificial sequence
  oligonucleotide
gcccactggctgccaagagg

SEQ ID NO: 6 - DNA, 20 bp, artificial sequence
  oligonucleotide
tctctcctcaccagcaccgt

SEQ ID NO: 7 - DNA, 20 bp, artificial sequence
  oligonucleotide
aaaggtctggagctggtagg

SEQ ID NO: 8 - DNA, 20 bp, artificial sequence
  oligonucleotide
gcgtgtccacctctaggacc

SEQ ID NO: 9 - DNA, 20 bp, artificial sequence
  oligonucleotide
ccagtgccaggtggacctgg

SEQ ID NO: 10 - DNA, 20 bp, artificial sequence
  oligonucleotide
ccagtattactgcacacgtc

SEQ ID NO: 11 - DNA, 20 bp, artificial sequence
  oligonucleotide
cctctggcttcgtcagaatc

SEQ ID NO: 12 - DNA, 20 bp, artificial sequence
  oligonucleotide
ggtggccttcagcaggagct

SEQ ID NO: 13 - DNA, 20 bp, artificial sequence
  oligonucleotide
catacaggacacgaagctcc

SEQ ID NO: 14 - DNA, 20 bp, artificial sequence
  oligonucleotide
catcctttagacacttgagc

SEQ ID NO: 15 - DNA, 20 bp, artificial sequence
  oligonucleotide
gctcctggcccgacagaggt

SEQ ID NO: 16 - DNA, 20 bp, artificial sequence
  oligonucleotide
gctaccacagtgatgatgac

SEQ ID NO: 17 - DNA, 20 bp, artificial sequence
  oligonucleotide
ttgtgtgttcggtttcatgg

SEQ ID NO: 18 - DNA, 20 bp, artificial sequence
  oligonucleotide
ggaggcgtggcttgtgtgtt

SEQ ID NO: 19 - DNA, 20 bp, artificial sequence
  oligonucleotide
cctgtcccgggataggttca

SEQ ID NO: 20 - DNA, 20 bp, artificial sequence
  oligonucleotide
cgaggaagaggccctgtccc

SEQ ID NO: 21 - DNA, 20 bp, artificial sequence
  oligonucleotide
tccactctgttcagtgtggc

SEQ ID NO: 22 - DNA, 20 bp, artificial sequence
  oligonucleotide
tctgactgaggacaatgccc

SEQ ID NO: 23 - DNA, 20 bp, artificial sequence
  oligonucleotide
taggtgtgcaggtaccatgg

SEQ ID NO: 24 - DNA, 20 bp, artificial sequence
  oligonucleotide
cctctcatcaggctagactt

SEQ ID NO: 25 - DNA, 20 bp, artificial sequence
  oligonucleotide
ccagttgtatgtcctcatgg

SEQ ID NO: 26 - DNA, 20 bp, artificial sequence
  oligonucleotide
gggcctcagcatacccaata

SEQ ID NO: 27 - DNA, 20 bp, artificial sequence
  oligonucleotide
atgctacacatgtctatgga

SEQ ID NO: 28 - DNA, 20 bp, artificial sequence
  oligonucleotide
gcccaagctggcatccgtca

SEQ ID NO: 29 - DNA, 20 bp, artificial sequence
  oligonucleotide
agtgcccaagctggcatccg

SEQ ID NO: 30 - DNA, 20 bp, artificial sequence
  oligonucleotide
gctccgtgaggccagagacc

SEQ ID NO: 31 - DNA, 20 bp, artificial sequence
  oligonucleotide
caggcactctcctgcagtgt

SEQ ID NO: 32 - DNA, 20 bp, artificial sequence
  oligonucleotide
gaaaggcaggttggccaatg

SEQ ID NO: 33 - DNA, 20 bp, artificial sequence
  oligonucleotide
ggtaatctctgaacctgtga

SEQ ID NO: 34 - DNA, 20 bp, artificial sequence
  oligonucleotide
gtccagacatgaccgctgag

SEQ ID NO: 35 - DNA, 20 bp, artificial sequence
  oligonucleotide
ctggagctgcaatagtgcaa

SEQ ID NO: 36 - DNA, 20 bp, artificial sequence
  oligonucleotide
tacacatacacacacacaca

SEQ ID NO: 37 - DNA, 20 bp, artificial sequence
  oligonucleotide
gctgaggtgggaggatcact

SEQ ID NO: 38 - DNA, 20 bp, artificial sequence
  oligonucleotide
ggtgtggtgttgtgagccta

SEQ ID NO: 39 - DNA, 20 bp, artificial sequence
  oligonucleotide
ctaacacaaaggaagtctgg

SEQ ID NO: 40 - DNA, 20 bp, artificial sequence
  oligonucleotide
cagtgcccaagctggcatcc

SEQ ID NO: 41 - DNA, 18 bp, artificial sequence
  oligonucleotide
cgagaggcggacgggacc

SEQ ID NO: 42 - DNA, 18 bp, artificial sequence
  oligonucleotide
cgggcgcctcggaagacc

SEQ ID NO: 43 - DNA, 18 bp, artificial sequence
  oligonucleotide
tggctgcagcttccgaga

SEQ ID NO: 44 - DNA, 18 bp, artificial sequence
  oligonucleotide
cccgcggctgctcacagg

SEQ ID NO: 45 - DNA, 18 bp, artificial sequence
  oligonucleotide
caggagaagccgaggaag

SEQ ID NO: 46 - DNA, 18 bp, artificial sequence
  oligonucleotide
gggaggtgccgccgccgc

SEQ ID NO: 47 - DNA, 18 bp, artificial sequence
  oligonucleotide
ccgggtccctggatgtgc

SEQ ID NO: 48 - DNA, 18 bp, artificial sequence
  oligonucleotide
cctccgaacggctgcctc

SEQ ID NO: 49 - DNA, 18 bp, artificial sequence
  oligonucleotide
tctcctcagcagccagag

SEQ ID NO: 50 - DNA, 18 bp, artificial sequence
  oligonucleotide
cgcttggctctggaccgc

SEQ ID NO: 51 - DNA, 18 bp, artificial sequence
  oligonucleotide
tcttctgcaggatggaaa

SEQ ID NO: 52 - DNA, 18 bp, artificial sequence
  oligonucleotide
ggataaatataggtcaag

SEQ ID NO: 53 - DNA, 18 bp, artificial sequence
  oligonucleotide
tcaatattgttcctgtat

SEQ ID NO: 54 - DNA, 18 bp, artificial sequence
  oligonucleotide
ttaaatttggcggtgtca

SEQ ID NO: 55 - DNA, 18 bp, artificial sequence
  oligonucleotide
caagatcttcacaaaagg

SEQ ID NO: 56 - DNA, 18 bp, artificial sequence
  oligonucleotide
attacaccagttcgtccc

SEQ ID NO: 57 - DNA, 18 bp, artificial sequence
  oligonucleotide
tgtctctggtccttactt

SEQ ID NO: 58 - DNA, 18 bp, artificial sequence
  oligonucleotide
acatagcgcctctgactg

SEQ ID NO: 59 - DNA, 18 bp, artificial sequence
  oligonucleotide
gaatatatcttcaccttt

SEQ ID NO: 60 - DNA, 18 bp, artificial sequence
  oligonucleotide
ggaagaactctactttga

SEQ ID NO: 61 - DNA, 18 bp, artificial sequence
  oligonucleotide
tgaagaatgtatttaccc

SEQ ID NO: 62 - DNA, 18 bp, artificial sequence
  oligonucleotide
ggttggctttgtctttat

SEQ ID NO: 63 - DNA, 18 bp, artificial sequence
  oligonucleotide
tgctagcctctggatttg

SEQ ID NO: 64 - DNA, 18 bp, artificial sequence
  oligonucleotide
tctggatcagagtcagtg

SEQ ID NO: 65 - DNA, 18 bp, artificial sequence
  oligonucleotide
tattttcatggtgtttta

SEQ ID NO: 66 - DNA, 18 bp, artificial sequence
  oligonucleotide
tgttcctataactggtaa

SEQ ID NO: 67 - DNA, 18 bp, artificial sequence
  oligonucleotide
gtgtcaaaaccctgtgga

SEQ ID NO: 68 - DNA, 18 bp, artificial sequence
  oligonucleotide
actggaataaaacgggaa

SEQ ID NO: 69 - DNA, 18 bp, artificial sequence
  oligonucleotide
acttcagttggtgacaga

SEQ ID NO: 70 - DNA, 18 bp, artificial sequence
  oligonucleotide
tagcaaaacctttcggaa

SEQ ID NO: 71 - DNA, 18 bp, artificial sequence
  oligonucleotide
aattatttcctttctgag

SEQ ID NO: 72 - DNA, 18 bp, artificial sequence
  oligonucleotide
taaatagctggagatggt

SEQ ID NO: 73 - DNA, 18 bp, artificial sequence
  oligonucleotide
cagattaataactgtagc

SEQ ID NO: 74 - DNA, 18 bp, artificial sequence
  oligonucleotide
ccccaatacagattcact

SEQ ID NO: 75 - DNA, 18 bp, artificial sequence
  oligonucleotide
attgttgctgtgtttctt

SEQ ID NO: 76 - DNA, 18 bp, artificial sequence
  oligonucleotide
tgtttcaagcccattctt

SEQ ID NO: 77 - DNA, 20 bp, artificial sequence
  oligonucleotide
aatcccagctactcgggagg

SEQ ID NO: 78 - DNA, 20 bp, artificial sequence
  oligonucleotide
aagcccttacttgcatgtct

SEQ ID NO: 79 - DNA, 20 bp, artificial sequence
  oligonucleotide
gcctgaagcactgaacagta

SEQ ID NO: 80 - DNA, 20 bp, artificial sequence
  oligonucleotide
cgagctattaccacagtatt

SEQ ID NO: 81 - DNA, 20 bp, artificial sequence
  oligonucleotide
agccaatgacaccatacctt

SEQ ID NO: 82 - DNA, 21 bp, artificial sequence
  oligonucleotide
agcttggaagacgatcagcaa

SEQ ID NO: 83 - DNA, 28 bp, artificial sequence
  oligonucleotide
aaactgctgaactattgtaggagagatg

SEQ ID NO: 84 - DNA, 25 bp, artificial sequence
  oligonucleotide
agatgccgtgtttgatggctccagc

SEQ ID NO: 85 - DNA, 22 bp, artificial sequence
  oligonucleotide
catagagaccccgttgcctaaa

SEQ ID NO: 86 - DNA, 22 bp, artificial sequence
  oligonucleotide
tggctatcttcttgcacattgc

SEQ ID NO: 87 - DNA, 23 bp, artificial sequence
  oligonucleotide
ctcctgcctgggaacaaccggaa

SEQ ID NO: 88 - DNA, 26 bp, artificial sequence
  oligonucleotide
aatggctaagtgaagatgacaatcat

SEQ ID NO: 89 - DNA, 25 bp, artificial sequence
  oligonucleotide
tgcacatatcattacaccagttcgt

SEQ ID NO: 90 - DNA, 30 bp, artificial sequence
  oligonucleotide
ttgcagcaattcactgtaaagctggaaagg

SEQ ID NO: 91 - DNA, 20 bp, artificial sequence
  oligonucleotide
tgcaggtattggtgagtcgg

SEQ ID NO: 92 - DNA, 22 bp, artificial sequence
  oligonucleotide
tccaaggctctaggtggtcatt

SEQ ID NO: 93 - DNA, 25 bp, artificial sequence
  oligonucleotide
tcgcagcttggatggccacttacct

SEQ ID NO: 94 - DNA, 19 bp, artificial sequence
  oligonucleotide
gaaggtgaaggtcggagtg

SEQ ID NO: 95 - DNA, 20 bp, artificial sequence
  oligonucleotide
gaagatggtgatgggatttc

SEQ ID NO: 96 - DNA, 20 bp, artificial sequence
  oligonucleotide
caagcttcccgttctcagcc

SEQ ID NO: 97 - DNA, 23 bp, artificial sequence
  oligonucleotide
acaatcccattgacattgaggtt

SEQ ID NO: 98 - DNA, 26 bp, artificial sequence
  oligonucleotide
tttgctctatttttagcttgtgtgct

SEQ ID NO: 99 - DNA, 30 bp, artificial sequence
  oligonucleotide
aaggaggttccccctactgagacagttcct

SEQ ID NO: 100 - DNA, 25 bp, artificial sequence
  oligonucleotide
tggcaactaacgtagaaactcaaca

SEQ ID NO: 101 - DNA, 23 bp, artificial sequence
  oligonucleotide
tgccaagagcatgaatacagaga

SEQ ID NO: 102 - DNA, 37 bp, artificial sequence
  oligonucleotide
acaactatagacttgctcattgttcagactgattgcc

SEQ ID NO: 103 - DNA, 23 bp, artificial sequence
  oligonucleotide
tgtgattgaaccacttcctgtca

SEQ ID NO: 104 - DNA, 27 bp, artificial sequence
  oligonucleotide
ggagtggtgttattttcagtaggtgaa

SEQ ID NO: 105 - DNA, 22 bp, artificial sequence
  oligonucleotide
tccaactcgggacgtggctaca

SEQ ID NO: 106 - DNA, 19 bp, artificial sequence
  oligonucleotide
gcaggcgggactatcagga

SEQ ID NO: 107 - DNA, 21 bp, artificial sequence
  oligonucleotide
agtttgccgaccagaccttct

SEQ ID NO: 108 - DNA, 24 bp, artificial sequence
  oligonucleotide
tccactgcctccatgatgcaagcc

SEQ ID NO: 109 - DNA, 19 bp, artificial sequence
  oligonucleotide
cgagaggcggacgggaccg

SEQ ID NO: 110 - DNA, 21 bp, artificial sequence
  oligonucleotide
cgagaggcggacgggaccgtt

SEQ ID NO: 111 - DNA, 21 bp, artificial sequence
  oligonucleotide
ttgctctccgcctgccctggc

SEQ ID NO: 112 - RNA, 20 nt, artificial sequence
  oligonucleotide
uuugucucugguccuuacuu

SEQ ID NO: 113 - DNA, 20 bp, artificial sequence
  oligonucleotide
ctgctagcctctggatttga

SEQ ID NO: 114 - DNA, 20 bp, artificial sequence
  oligonucleotide
tccagcactttcttttccgg

SEQ ID NO: 115 - RNA, 20 nt, artificial sequence
  oligonucleotide
uuugucucugguccuuacuu

SEQ ID NO: 116 - RNA, 20 nt, artificial sequence
  oligonucleotide
uuuaucgcuucucguugcuu

SEQ ID NO: 117 - DNA, 21 bp, artificial sequence
  oligonucleotide
ccaauccagaggcuagaagtt

SEQ ID NO: 118 - DNA, 21 bp, artificial sequence
  oligonucleotide
ttgguuaggucuccgaucuuc

SEQ ID NO: 119 - DNA, 21 bp, artificial sequence
  oligonucleotide
ttguuuaggccuucgaucguc

SEQ ID NO: 120 - DNA, 21 bp, artificial sequence
  oligonucleotide
cuaaaccggaugccagaagtt

SEQ ID NO: 121 - DNA, 21 bp, artificial sequence
  oligonucleotide
ttgauuuggccuacggucuuc

(19) United States
(12) Patent Application Publication
(10) Pub. No.: US 2004/0137471 A1
(43) Pub. Date: Jul. 15, 2004

(54) Efficient reduction of target RNA's by single-and double-stranded oligomeric compounds

(76) Inventors: Timothy Vickers, Oceanside, CA (US); Seongjoon Koo, Carlsbad, CA (US); C. Frank Bennett, Carlsbad, CA (US); Stanley T. Crooke, Carlsbad, CA (US); Nicholas M. Dean, Olivenhain, CA (US); Brenda F. Baker, Carlsbad, CA (US)

Correspondence Address: COZEN O'CONNOR, P.C., 1900 MARKET STREET, PHILADELPHIA, PA, 19103-3508, US

(21) Appl. No.: 10/664,639

(22) Filed: Sep. 18, 2003

Related U.S. Application Data
(60) Provisional application No. 60/411,780, filed on Sep. 18, 2002.

(51) Int. Cl. C12Q 1/68; C07H 21/02

(52) U.S. Cl. 435/6; 536/23.1

(57) ABSTRACT

The present invention provides, inter alia, methods of selecting a single-stranded oligomeric compounds for inhibiting RNA expression, methods of generating double-stranded oligomeric compounds, methods of identifying optimized double-stranded oligomeric compounds, methods of selecting optimized single-stranded oligomeric compounds, methods of selecting optimized double-stranded oligomeric compounds, methods of identifying multifunctional oligomeric compounds, methods for optimizing target region selection for modulation of RNA expression, methods of optimizing expression modulation of RNA, and the like. The present invention further provides oligomeric compounds, 8-80 nucleobases in length targeted to a target RNA, wherein said oligomeric compound hybridizes to said target RNA and inhibits RNA levels by at least 50% in both single-stranded and double-stranded forms, and multifunctional oligomeric compounds.

### CROSS-REFERENCE TO RELATED APPLICATIONS

The present application claims priority of U.S. Application Serial No. 60/411,780 filed Sep. 18, 2002, which is hereby incorporated by reference in its entirety.

### FIELD OF THE INVENTION

The present invention provides, inter alia, compositions and methods for modulating the levels of gene products. The present invention also provides methods for selecting and designing optimized oligomeric compounds.

### BACKGROUND OF THE INVENTION

RNA interference (RNAi) and post-transcriptional gene silencing (PTGS) have become powerful and widely used tools for the analysis of gene function in invertebrates and plants [Fraser et al. (2000), Nature, 408,325-330; Gönczy et al. (2000), Nature, 408(331-336)]. Introduction of double-stranded RNA (dsRNA) into the cells of these organisms leads to the sequence-specific degradation of homologous gene transcripts. The long double-stranded RNA molecules are reduced to small 21-23 nucleotide interfering RNAs (siRNAs) by the action of an endogenous ribonuclease, dicer. (Bernstein et al. (2001), Nature, 409,363-366; Grishok et al. (2000), Science, 287 (5462), 2494-7; Zamore et al. (2000), Cell, 101(1), 25-33; Knight, S. W. and B. L. Bass. (2001), Science, 293(5538), 2269-2271).

In mammalian cells, initial attempts to use long double-stranded RNA appeared to fail to induce specific inhibition of gene expression (Tuschl et al. (2000), Genes & Development, 13,3191; Caplen, N. J., J. Fleenor, and A. F. A. Morgan. (2000), Gene, 252(1-2), 95-105; Oates et al. (2000), Dev. Biol., 224, 20-28). The large double-stranded RNA molecules were found to promote a global change in gene expression, obscuring any gene specific silencing. This reduction in global gene expression is thought to be mediated in part, through activation of double-stranded RNA-activated protein kinase (PKR) which phosphorylates and inactivates the translation factor eIF2α (Der et al (1997), Proc. Natl Acad. Sci. USA, 94,3279-3283). Recently it has been shown that transfection of synthetic 21-nucleotide siRNA duplexes into mammalian cells does not elicit the PKR response allowing effective inhibition endogenous genes in a sequence-specific manner (Elbashir, S. M., et al. (2001), Nature, 411(6836), 494-8; Caplen et al. (2001), Proc. Natl. Acad. Sci. USA, 98,9742-9747). These siRNA duplexes appear to be too short to trigger the nonspecific dsRNA responses, but they still promote degradation of complementary RNA sequences.

siRNAs were initially employed in mammalian cells targeted to non-human transgene transcripts like green fluorescent protein, chloramphenicol acetyl transferase, and luciferase (Elbashir, S. M., et al. (2001), Nature, 411(6836), 494-8; Caplen et al. (2001), Proc. Natl. Acad. Sci. USA, 98,9742-9747). More recently siRNA molecules have been used against a variety of endogenous expressed mammalian proteins (Holen, T., et al. (2002), Nucleic Acids Res, 30(8), 1757-1766; Martins, L. M., et al. (2002), J. Biol. Chem, 277(1), 439-444; Novina, C. D., et al. (2002), Nature Medicine, 8(7), 681-686; Harborth, J., et al. (2001), Journal of Cell Science, 114, 4557-4565; Kufer, T. A., et al. (2001), Journal of Cell Biology, 158(4), 617-623; Prasanth et al. (2002), Science, 297(5583), 1026-1031). For example a siRNA molecule was used to inhibit the expression of the serine protease Omi/HtrA2 (Martins, L. M., et al. (2002), J. Biol. Chem, 277(1), 439-444). The siRNA targeting Omi/HtrA2 resulted in a significant decrease in Omi/HtrA2 expression and a concomitant abrogation of the apoptotic response to UV exposure. These results appeared to demonstrate that siRNA molecules are useful tools to determine the function of genes in mammalian cell cultures.

Recent models of siRNA activity, based upon studies in Drosophila, propose that they work by a novel antisense mechanism. The short double-stranded RNA molecules bind to a protein complex, termed RNA-induced silencing complex (RISC) which contains a helicase that unwinds the two strands of RNA molecules, allowing the antisense strand to bind to the targeted RNA molecule (Zamore, P. D., et al. (2000), Cell, 101(1), 25-33; Elbashir, S. M., et al. (2002), Methods, 26(2), 199-213; Zamore, P. D. (2002), Science, 296 (5571), 1265-1269). An endonuclease, which is also a component of the RISC complex, enzymatically hydrolyzes the target RNA at the site where the antisense strand is bound. It is unknown whether the antisense RNA molecule is also hydrolyzed or recycles and binds to another RNA molecule.

There are multiple mechanisms by which short oligonucleotides can be used to modulate gene expression in mammalian cells (Crooke, S. T. (1999), Biochim, Biophys. Acta., 1489(1),30-42). For example, it has been shown that single-stranded oligoribonucleotides bound to a specific mRNA, serving as a substrate for a novel double-stranded RNase (Wu, H., et al. (1998), Journal of Biological Chemistry, 273(5), 2532-2542). In this case the oligoribonucleotide was chemically modified with a phosphorothioate linkage to provide nuclease resistance and could be further modified with 2-O-methyl residues on the ends, but appeared to be inactive if uniformly modified. The most commonly exploited antisense mechanism for single-stranded oligonucleotides is RNase H dependent degradation of the targeted RNA. RNase H is a ubiquitously expressed endonuclease that recognizes a DNA-RNA heteroduplex, hydrolyzing the RNA strand. Thus siRNA differs from the most widely used antisense mechanism by utilizing a double-stranded RNase, instead of RNase H as the terminating mechanism.

Reports in which siRNA was compared to single-stranded antisense approaches to gene knockdown have appeared to indicate that the siRNA is more potent and effective than traditional antisense approaches (Zamore, P. D., et al. (2000), Cell, 101(1), 25-33; Lee, N. S., et al. (2002), Nature Biotechnology, 20,500-505; Caplen, N. J., et al. (2001), Proc Natl Acad Sci USA, 98,9742-9747). However, the antisense molecules used in these experiments were single-stranded RNA, which are rapidly degraded and do not recruit RNase H to cleave the target. Phosphorothioate oligodeoxynucleotides are first-generation antisense agents that have been widely used to modulate gene expression in cell based assays, in animal models and in the clinic. The phosphorothioate modification dramatically increases the nuclease resistance of the oligonucleotide and still supports RNase H activity (Eckstein, F. (2000), Antisense Nucleic Acid Drug Dev, 10(2), 117-121). Further improvements to phosphorothioate oligodeoxynucleotides, have been made resulting in second-generation oligonucleotides such as 2′-O-methyl or 2′-O-methoxyethyl modifications (Monia, B. P., et al. (1993), Journal of Biological Chemistry, 268(19), 14514-22; Agrawal, S., et al. (1997), Proc Natl Acad Sci USA, 94(6), 2620-2625; McKay, R. A., et al. (1999), J Biol Chem, 274(3), 1715-22). The 2′-O-methoxyethyl modification is particularly attractive as it increases the potency of the oligodeoxynucleotide, further increases nuclease resistance, decreases toxicity and increases oral bioavailability in the RNAse H antisense mechanism (Baker, B. F., et al. (1997), J. Biol. Chem., 272(18), 11994-12000; Henry, S., et al. (2000), J Pharmacol Exp Ther, 292(2), 468-479; Geary, R. S., et al. (2001), Journal of Pharmacological and Experimental Therapeutics, 296(3), 898-904; Geary, R. S., et al. (2001), Journal of Pharmacology and Experimental Therapeutics, 296(3), 890-897; Yu, R. Z., et al. (2001), J Pharmacol Exp Ther, 296(2), 388-395).

### SUMMARY OF THE INVENTION

The present invention provides, inter alia, methods of identifying a multifunctional oligomeric compound to modulate expression of RNA. The methods comprise (a) contacting a target RNA with one or more double-stranded oligomeric compounds hybridizable to one or more target regions of the RNA and identifying double-stranded oligomeric compounds which inhibit target RNA levels by at least 50%; (b) contacting the target RNA with an antisense strand of the modulating double-stranded oligomeric compound and determining whether the antisense strand inhibits target RNA levels by at least 50%; and (c) identifying antisense strand and double-stranded oligomeric compound that inhibit target RNA levels by at least 50% as “multifunctional” oligomeric compounds. In some embodiments, the present invention provides the multifunctional oligomeric compounds so identified. In some embodiments the multifunctional oligomeric compound inhibits target RNA levels by at least 80%.

In some embodiments the target region is identified by a single-stranded oligomeric gene walk across the target RNA. In some embodiments the target region is identified by secondary structure analysis of the target RNA.

In some embodiments the target region is at least a portion of an induced gene. In some embodiments the target region is at least a portion of a constitutive gene. In some embodiments the target region is localized to the 3′UTR, the 5′UTR, an intron:exon boundary, an exon:exon boundary, a start region or a coding region of the RNA. In some embodiments the target region is localized to an intronic portion of a gene. In some embodiments the target region is localized to an exon. In some embodiments the target region overlaps the intron/exon boundary with 5-10 nucleotides on either side of the boundary.

In some embodiments the oligomeric compound is an antisense oligonucleotide. In some embodiments the oligomeric compound has at least one modification of the base, sugar or internucleoside linkage. In some embodiments the oligomeric compound has a modification at the 2′ position of at least one sugar. In some embodiments oligomeric compound is from about 12 to about 50 nucleotides in length. In some embodiments the oligomeric compound comprises at least four consecutive 2′-hydroxyl ribonucleosides and at least one modified nucleoside; said modified nucleoside adapted to modulate at least one of; binding affinity or binding specificity of said oligomeric compound. In some embodiments the oligomeric compound is a gapmer, a hemimer, or a chimeric compound. In some embodiments the oligomeric compound comprises at least six consecutive nucleosides with 2′ modifications.

The present invention also provides methods for optimizing target region selection for modulation of RNA expression. The methods comprise (a) contacting double-stranded oligomeric compounds with one or more regions of a target RNA and identifying target regions which, when contacted with the one or more double-stranded oligomeric compounds, result in inhibition of target RNA levels of at least 50%; (b) contacting single-stranded oligomeric compounds with target regions that were inhibited at least 50% by double-stranded oligomeric compounds and identifying regions which, when contacted with the single-stranded oligomeric compounds, result in inhibition of target RNA levels of at least 50%; and (c) identifying those target regions that are modulated by at least one double-stranded oligomeric compound and at least one single-stranded oligomeric compound as “optimized” target regions. In some embodiments target RNA levels are inhibited by at least 80% by single-stranded oligomeric compounds and double-stranded oligomeric compounds.

The present invention further provides methods of optimizing modulation of RNA comprising contacting a target RNA with at least two oligomeric compounds hybridizable to a target region of the target RNA wherein at least two oligomeric compounds each inhibit RNA levels by at least 50% when tested individually.

The present invention also provides methods of optimizing target regions of RNA comprising contacting a target RNA comprising a target region with oligomeric compounds hybridizable with the target region; and identifying target regions as “optimized” when two or more of the oligomeric compounds inhibit target RNA levels by at least 50%. In some embodiments at least one of the oligomeric compounds comprise a double-stranded region. In some embodiments, the target regions are “optimized” when two or more of the oligomeric compounds inhibit target RNA levels by at least 80%.

The present invention further provides methods of selecting a target region of a gene comprising (a) contacting a target RNA comprising at least one target region with a plurality of oligomeric compounds, each compound hybridizable with a target region. The oligomeric compounds include at least one siRNA oligomeric compound and at least one ASO oligomeric compound siRNA and ASO oligomeric compounds which inhibit RNA levels by at least 60% for the target region are identified; and target regions are selected when there is a significant association between siRNA oligomeric compounds which inhibit RNA levels by at least 60% and ASO oligomeric compounds which inhibit RNA levels by at least 80% for the target region. In some embodiments, determining “significant association” is performed using a ROC analysis.

The present invention also provides methods of selecting an optimized single-stranded oligomeric compound comprising (a) contacting a target RNA with one or more double-stranded oligomeric compounds; (b) identifying one or more double-stranded oligomeric compounds which inhibit target RNA levels by at least 50%; and (c) selecting the strand of the double-stranded oligomeric compound that hybridizes to the target RNA as the optimized single-stranded oligomeric compound. In some embodiments target RNA levels are inhibited by at least 80%.

The present invention still further provides methods of selecting an optimized double-stranded oligomeric compound comprising (a) contacting a target RNA with one or more single-stranded oligomeric compounds; (b) identifying single-stranded oligomeric compounds which inhibit target RNA levels by at least 50%; and (c) hybridizing a complementary single-stranded oligomeric compound to the single-stranded oligomeric compound to yield an “optimized” double-stranded oligomeric compound.

The present invention also provides methods of selecting a single-stranded oligomeric compound comprising (a) contacting a target RNA with double-stranded oligomeric compounds; (b) identifying double-stranded oligomeric compounds which inhibit target RNA levels by at least 50%; and (c) selecting the strand of the identified double-stranded oligomeric compound which is complementary to the target RNA as the selected single-stranded oligomeric compound.

The present invention further provides methods of generating a double-stranded oligomeric compound comprising (a) contacting a target RNA with single-stranded oligomeric compounds; (b) identifying single-stranded oligomeric compounds which inhibit target RNA levels by at least 50%; and (c) hybridizing a complementary single-stranded oligomeric compound to the single-stranded oligomeric compound that inhibits target RNA levels by at least 50%, yielding a double-stranded oligomeric compound.

The present invention provides methods of identifying optimized double-stranded oligomeric compounds comprising (a) cloning target regions from a target RNA into a vector/plasmid construct; (b) transfecting the vector/plasmid into a cell; (c) contacting a cell transfected with the vector/plasmid with double-stranded oligomeric compounds having one strand hybridizable to said target region; and, (d) identifying the double-stranded oligomeric compounds which inhibit target RNA levels by at least 50%.

The present invention also provides oligomeric compounds, 8-80 nucleobases in length, targeted to a target RNA, wherein the oligomeric compound specifically hybridizes to the target RNA and inhibits RNA levels by at least 50% in both single-stranded and double-stranded forms. In some embodiments RNA levels are measured in A549 cells.

The present invention further provides oligomeric compounds, 8-80 nucleobases in length targeted to a target RNA. The oligomeric compounds have at least 80% sequence homology to the complement of the target RNA and inhibit RNA levels by at least 60% in both single-stranded and double-stranded forms. In some embodiments the sequence homology between the oligomeric compound and the complement of the target RNA is at least 90%. In some embodiments the oligomeric compounds have at least 2 mismatches as compared to the complement of the target RNA. In some embodiments the mismatches are internal or external base mismatches. In some embodiments no more than two of the four 3′-most nucleotides of the oligomeric compound are mismatches. In some embodiments the oligomeric compound has an IC₅₀ no greater than 100 nM or no greater than 10 nM.

In some embodiments the oligomeric compound has alternating linkages. In some embodiments the oligomeric compound has alternating modifications. In some embodiments every second nucleotide in the antisense strand of the double-stranded oligomeric compound is modified. In some embodiments the first modified nucleotide is the 5′-most nucleotide of the oligomeric compound. In some embodiments the modifications are 2′ modifications selected from the group consisting of 2′-O alkyl, 2′-O-methoxyethyl, 2′-methoxyethoxy, 2′-dimethylaminooxyethoxy, 2′-dimethylaminoethoxyethoxy, 2′-methoxy, 2′-aminopropoxy, 2′-allyl, 2′-O-allyl (2′-O-CH₂—CH═CH₂), or 2′-fluoro.

In some embodiments the oligomeric compound comprises a first segment; a second segment; and, a third segment which is located between the first and second segments and comprises three or four nucleobases, wherein the first and second segments each have at least one modified nucleobase. In some embodiments the third segment has no modified nucleobases or modified linkages. In some embodiments the first and second segments each comprise at least one modified linkage/modification. In some embodiments the oligomeric compound comprises at least seven 2′-O-methyl substitutions at the 3′-terminus of the oligomeric compound. In some embodiments the oligomeric compound has at least six mismatches as compared to the complement of the target RNA.

### DETAILED DESCRIPTION OF THE INVENTION

The present application compares oligonucleotides that work by a siRNA mechanism to optimized first- and second-generation antisense oligonucleotides that work by an RNase H dependent mechanism. Active siRNAs and homologous RNase H-dependent oligonucleotides were evaluated for relative potency, efficacy, duration of action, potency, specificity and site of action within the cell to determine advantages for the different antisense strategies in cell based assays. In some embodiments the results suggest that in human cell culture based assays, double-stranded oligoribonucleotides that work by siRNA mechanism exhibit similar potency efficacy and duration of action as RNase H-dependent oligonucleotides. Finally, siRNA and RNase H-dependent oligonucleotides appear to work in different cellular compartments.

Although RNAi is thought to work through an antisense mechanism, for the sake of convenience, the term “siRNA” will be used to refer to RNAi oligonucleotides while the term “ASO” will be used to refer to RNAse H-dependent antisense oligonucleotides.

There are multiple mechanisms by which synthetic oligonucleotides can be used to regulate gene expression in mammalian cells (Crooke, S. T. (1999) Molecular mechanisms of action of antisense drugs. Biochim, Biophys. Acta., 1489(1), 30-42). By far, the most successful strategy to date has been to design oligonucleotides to hybridize to a target RNA by Watson-Crick base pairing rules, i.e. antisense oligonucleotides. There are two general mechanisms by which antisense oligonucleotides can be used to regulate gene expression; occupancy-only mediated mechanisms and degradation of targeted RNA. Examples of occupancy-only mechanisms include, inhibition of translation (Baker, B. F., et al. (1997). J. Biol. Chem., 272(18), 11994-12000; Helene, C. and J.-J. Toulme. (1990) Biochim. Biophys. Acta, 1049, 99-125), modulation of pre-mRNA splicing (Kole, R. and D. Mercatante, Pre-mRNA Splicing as a Target for Antisense Oligonucleotides, in Antisense Technology: Principles, Strategies and Applications, S. T. Crooke, Editor. 2001, Marcel Dekker, Inc.: New York. p. 517-539; Taylor, J. K., et al. (1999) Nat. Biotech., 17(11),1097-1100) or modulation of polyadenylation (Vickers et al. (2001) Nucleic Acids Research, 29(6), 1293-1299). There are several endogenous enzymes that can be exploited to promote targeted cleavage of RNAs in cells, as well as chemical means to promote RNA hydrolysis. The most widely exploited mechanism is RNase H mediated cleavage of targeted RNA. RNase H is a ubiquitously expressed cellular enzyme that hydrolyzes the RNA strand of an RNA-DNA heteroduplex. As such the antisense oligonucleotide should contain at least five consecutive DNA molecules to support RNase H activity in human cells (Monia, B. P., et al.

Journal of Biological Chemistry, 268(19), 14514-22). There are additional RNases present in mammalian cells that can be exploited. As an example, we reported that a single-stranded phosphorothioate modified RNA molecule can promote selective loss of ha-ras in human cells (Wu, H., et al. (1998) Journal of Biological Chemistry, 273(5), 2532-2542). Similar to RNase H, this double-stranded RNase required a minimal gap of four consecutive ribonucleotides. Ribozymes and DNAzymes are antisense molecules that possess autocatalytic activity, resulting in cleavage of the targeted RNA and have been used to inhibit gene expression in mammalian systems (Cech, T. R. (1992) Curr. Opin. Struct. Biol., 2, 605-609; Flory, C. M., et al. (1996) Proc Natl Acad Sci USA, 93(2), 754-8; Santoro, S. W. and G. F. Joyce. (1997) Proc. Natl. Acad. Sci. USA, 94,4262-4266). Several investigators have attempted to modify the antisense oligonucleotide with a chemical catalyst to generate artificial nucleases (Hall, J., D. Hisken, and R. Haner. (1996); Nucleic Acids Research, 24(18), 3522-3526; Haner, R. and J. Hall. (1997) Antisense Nucleic Acid Drug Dev, 7(4), 423-430; Baker, B. F., et al. (1999) Nucleic Acids Research, 27(6), 1547-51).

Post-transcriptional gene silencing by double-stranded RNA molecules, RNA interference, has proven to be a very effective and novel antisense mechanism for investigation of gene function in plants and other model systems (Zamore, P. D. (2002) Science, 296(5571), 1265-1269). In non-vertebrate organisms, introduction of RNA molecules greater than 50 nucleotides in length produces a specific reduction of target RNA to levels that were not detectable by the methods employed. Furthermore, some researchers have reported that the effects last for multiple generations as the RNAi molecules and have speculated that they appear to be amplified by an RNA dependent RNA polymerase (Fire, A., et al. (1998) Nature, 391, 806-811; Lipardi et al. (2001) Cell, 107, 297-307; Nykänen et al. (2001) Cell, 107,309-321). Studies investigating the mechanism of RNA interference revealed that the long double-stranded RNA molecules were cleaved to short 21 to 25 nucleotide fragments by a double-stranded RNase III enzyme, Dicer (Zamore, P. D., et al. (2000) Cell, 101(1), 25-33; Hamilton, A. J. and D. C. Baulcombe. (1999) Science, 286(5441), 950-2). The small RNA fragments, in turn, dissociate in the presence of an RNA helicase, with the antisense strand binding to the target RNA, where it induces cleavage of the target RNA by an uncharacterized RNase. In some species, such as C. elegans, the RNA fragments can also serve as primers for an RNA dependent RNA polymerase resulting in generation of a new long double-stranded RNA molecule (Sijen, T., et al. (2001) Cell, 107(4), 465-76). Thus, a limited number of RNAi molecules can be amplified, generating larger numbers of interfering RNA molecules in cells, augmenting the potency of RNAi molecules.

In contrast to Drosophilia, C. elegans and other non-vertebrate species, introduction of long double-stranded RNA molecules in most mammalian cultured cells results in a generalized suppression of protein synthesis (Tuschl, T., et al. (2000) Genes & Development, 13,3191; Caplen et al. (2000) Gene, 252(1-2), 95-105; Oates et al. (2000) Dev. Biol., 224,20-28), which has been attributed to activation of RNA-dependent protein kinase and subsequent phosphorylation of eIF2α and other potential substrates (Der et al. (1997) Proc. Natl. Acad. Sci. USA, 94,3279-3283; Jammi, N. V. and P. A. Beal. (2001) Nucleic Acids Research, 29(14), 3020-3029). Subsequently it was reported that RNAi cleavage products, small interfering RNA fragments (siRNA), could be added to cellular extracts or transfected into mammalian cells and induce specific cleavage of the target RNA molecule (Elbashir, S. M., et al. (2001) Nature, 411(6836), 494-8; Caplen, N. J., et al. (2001) Proc Natl Acad Sci USA, 98, 9742-9747). These small RNA fragments do not appear to activate double strand RNA-dependent protein kinase.

Small interfering RNAs have been gaining widespread acceptance as a valuable tool for inhibiting gene expression in mammalian cells. In that siRNA is an antisense mechanism resulting in loss of target RNA, siRNA was compared to the most commonly used antisense mechanism of action, RNase H mediated degradation of target RNA (Crooke, S. T. (1999) Biochim, Biophys. Acta., 1489(1), 30-42). In both cases, a single-stranded oligonucleotide molecule binds to the target RNA by Watson-Crick base pairing. The RNase that recognizes the duplex formed by the siRNA molecule has not been identified to date, however, the substrate specificity suggest that it is a double strand specific RNase (Elbashir, S. M., et al. (2001) EMBO J, 20,6877-6888). It has recently been reported that siRNA efficacy is highly dependent upon target position (Holen, T., et al. (2002) Nucleic Acids Res, 30(8), 1757-1766). Since RNase H-dependent oligonucleotides are also known to be dependent upon target position (Chiang, M. Y., et al. (1991) Journal of Biological Chemistry, 266(27), 18162-71; Johansson, H. E., et al. (1994) Nucleic Acids Research, 22,4591-4598; Bennett, C. F., et al. (1994) Nucleic Acids Research, 22(15), 3202-3209) we sought to determine if active RNase H oligonucleotide binding sites would also be active sites for siRNA. In 3 of 4 cases (ISIS 5132, ISIS 116847, ISIS 16009) active siRNAs that targeted a site previously shown to be a good target site for RNase H-dependent oligonucleotides, showed activity comparable to that of the RNase H oligonucleotide. In the single case where the siRNA did not show activity comparable to that of the RNase H oligonucleotide (ISIS 2302), activity was not obtained when the siRNA was designed based upon the method recommended by Elbashir et. al (Elbashir, S. M., et al. (2002) Methods, 26(2), 199-213). Analysis of oligonucleotide screens against both CD54 and PTEN appears to confirm that target position is an important factor in determining siRNA activity. There was a significant degree of correlation between the RNase H-dependent oligonucleotides and siRNA screens, suggesting that if a site is available for hybridization to an ASO it is also available for hybridization and cleavage by the siRNA complex.

Since the structure of the mRNA target appears to be an important factor in determining ASO efficacy (Eckardt, S., P. Romby, and G. Sczakiel. (1997) Biochemistry, 36(42), 12711-12721; Fedor, M. J. and O. C. Uhlenbeck. (1990) Proc Natl Acad Sci USA, 87, 1668-1672), it might also play a role in siRNA activity. To address this issue ASOs and siRNA were evaluated for activity in a system in which mRNA with known structures were targeted (Vickers et al. (2000) Nucleic Acids Res., 28(6), 1340-1347). While the potency of the siRNA oligonucleotides and ASO oligonucleotides was comparable against the unstructured target, neither reduced luciferase expression even at the highest doses tested.

To determine if siRNA molecules were more potent or effective inhibitors of gene expression, an optimized siRNA molecule was compared to an optimized second-generation antisense molecule targeting either PTEN or CD54. In both cases, the oligonucleotides working by either antisense mechanism exhibited similar potencies. Additionally, both types of oligonucleotides inhibited the respective target genes by greater than 90%. Both siRNA and the RNase H-dependent oligonucleotides gave similar duration of action in cultured cells, both showing a gradual recovery of mRNA expression over four to six days. This loss of activity may be attributed to dilution of oligonucleotide concentration as cells divide. This data also appears to argue against the presence of a propagative system in mammalian cells similar to that observed in Drosophila and C. elegans (Lipardi et al. (2001) Cell, 107, 297-307; Sijen, T., et al. (2001) Cell, 107(4), 465-76), which amplifies siRNA based silencing over time. From this comparison, the onset of the RNase H-dependent activity appears to be slightly earlier than that of the siRNA. This may be a result of differences resulting from the ASO acting in the nucleus on the pre-mRNA while siRNA acts cytoplasmically on the mRNA only.

The effect of mismatches for both the RNase H-dependent oligonucleotides and siRNAs were also compared. The fidelity for perfect base-pair matches in ASO-based technologies has been suggested to be dependent on the position of the mismatches (Lima, W. F. and S. T. Crooke. (1997) Binding Affinity and Specificity of Escherichia coli RNase H1: Impact on the Kinetics of Catalysis of Antisense Oligonucleotide-RNA Hybrids. Biochemistry, 36(2), 390-398). Oligonucleotides were designed with internal or external 2-base mismatches and the effects on mRNA reduction were compared. Activity was lost when two-base mismatches were made in the central domain of either the RNase H-dependent oligonucleotide or siRNA. When mismatches were placed near the ends of the sequence, activity was reduced. The loss of activity was greater for the RNase H-dependent oligonucleotide than the siRNA, but not significantly so.

Selective knock-down of alternatively spliced mRNA products may be achieved simply by targeting the isoform-specific exon or exon:exon borders as found in the mature spliced product.

The activity of several siRNAs and homologous ASOs targeted to intron sequence were evaluated. Any observed reduction in the target gene expression may be the result of nuclear localization of the activity as the intron sequence should only be available for hybridization in the nucleus of the cell. While all of the RNase H-dependent oligonucleotides demonstrated significant and specific reduction of the targeted message, none of the siRNAs did. Although not wishing to be bound to the theory, the data supports the hypothesis that siRNA activity is predominantly, if not exclusively, cytoplasmic.

Optimized siRNA and RNase H-dependent oligonucleotides appear to behave similarly in terms of potency, maximal effects, specificity, and duration of action and efficiency. They do appear to differ significantly with respect to cellular location of activity, with siRNA promoting cleavage of mature mRNA and RNase H-dependent oligonucleotides promoting cleavage of pre-mRNA. There are specific instances where it may be advantageous to selectively target pre-mRNA or mature mRNA, such as modulation of RNA maturation or selective inhibition of alternative spliced variants, respectively. However, for cell based assays, both strategies appear to be valid.

Definitions

Various definitions are made throughout this document. Most words have the meaning that would be attributed to those words by one skilled in the art. Words specifically defined either below or elsewhere in this document have the meaning provided in the context of the present invention as a whole and as typically understood by those skilled in the art.

In the context of the present invention, “modulation” means either an increase (stimulation) or a decrease (inhibition) in the expression of a gene. In some embodiments, “modulation” is inhibition of the gene of interest.

As used herein, the term “contacting” means bringing together, either directly or indirectly, a compound into physical proximity to another compound. The compounds can be present in any number of buffers, salts, solutions, etc. Contacting includes, for example, placing the compound into a beaker, microtiter plate, cell culture flask, or a microarray, such as a gene chip, or the like.

In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. In some embodiments modified or substituted oligonucleotides have desirable properties over native forms including, for example, enhanced cellular uptake, enhanced affinity for a target nucleic acid and increased stability in the presence of nucleases.

As used herein, the phrase “siRNA oligonucleotide” refers to a RNAi oligonucleotide.

As used herein, the phrase “ASO oligonucleotide” refers to a RNAse H-dependent antisense oligonucleotide.

In some embodiments, oligomeric compounds comprises from about 5 to 100 nucleobases. In some embodiments, oligomeric compounds comprise from about 8 to about 50 nucleobases (i.e. from about 8 to about 50 linked nucleosides), and from about 12 to about 30 nucleobases. The present invention is also intended to comprehend other oligomeric compounds from about 8 to about 50 nucleobases in length which hybridize to the nucleic acid target and which inhibit expression of the target. Such compounds include ribozymes, external guide sequence (EGS) oligonucleotides (oligozymes), and other short catalytic RNAs or catalytic oligonucleotides. In some embodiments, oligomeric compounds are single or double-stranded. In some embodiments of the present invention, the oligomeric compounds comprise one or more double-stranded regions. In some embodiments the double-stranded region is a hairpin structure. In some embodiments, the oligomeric compounds of the present invention are compounds of about 15-30 nucleotides in length comprising a central hybridization region of about 19 nucleotides.

The term “region” refers to a physically contiguous portion of the primary structure of a biomolecule. In the case of proteins, a region is defined by a contiguous portion of the amino acid sequence of that protein.

As used herein, the term “inducible gene” refers to a gene which can be upregulated above basal levels in response to external stimuli. These stimuli include, but are not limited to, contact with viruses, bacteria, or other infective organisms, chemical contact, UV exposure, heat, growth factors, cytokines, chemokines, stressors such as wounding, ions, steroids and combinations thereof. Examples of inducible genes include without limitation, CD54, TRADD, inflammatory pathway components, NK4, SAA complement C3, prosaposin, b-APP, t-Tgase, CDK inhibitors; genes associated with Alzheimer's disease, amylodosis, arthritis, atherosclerosis, Erythropoietin, VEGF, glucose transporters, glycolytic enzymes, PSA, human glandular kallikrein, NKX3, ornithine decarboxylase, and the like.

In the context of the present invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that in some instances the sequence of an antisense compound need not be 100% complementary to that of its target nucleic acid to be specifically hybridizable. An oligomeric compound is specifically hybridizable when binding of the compound to the target DNA or RNA molecule interferes with the normal function of the target DNA or RNA to cause a modulation of activity, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

For example, typical highly stringent hybridization conditions are as follows: hybridization at 42° C. in a solution comprising 50% formamide, 1% SDS, 1 M NaCl, 10% Dextran sulfate and washing twice for 30 minutes each wash at 60° C. in a wash solution comprising 0.1 X SSC and 1% SDS. Those skilled in the art understand that conditions of equivalent stringency can also be achieved through varying temperature and buffer, or salt concentration as described by Ausubel et al. (Protocols in Molecular Biology, John Wiley & Sons (1994), pp. 6.0.3 to 6.4.10). Modifications in hybridization conditions can be empirically determined or precisely calculated based on the length and the percentage of guanosine/cytosine (GC) base pairing of the probe. Hybridization conditions can be calculated as described in, for example, Sambrook et al., (Eds.), Molecular Cloning: A Laboratory Manual, Cold Spring Harbor Laboratory Press: Cold Spring Harbor, N.Y. (1989), pp. 9.47 to 9.51.

As used herein, “moderate stringency hybridization conditions” means hybridization at 55° C. with 6 X SSC containing 0.5% SDS; followed by two washes at 37° C. with 1 X SSC.

As used herein, the term “percent homology” and its variants are used interchangeably with “percent identity” and “percent similarity.”

Percent homology can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity or complementarity, between the oligomeric compound and target is between about 50% to about 60%. In some embodiments, homology, sequence identity or complementarity, is between about 60% to about 70%. In some embodiments, homology, sequence identity or complementarity, is between about 70% and about 80%. In some embodiments, homology, sequence identity or complementarity, is between about 80% and about 90%. In some embodiments, homology, sequence identity or complementarity, is about 90%, about 92%, about 94%, about 95%, about 96%, about 97%, about 98%, or about 99%.

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

As used herein, the term “multifunctional” refers to an oligomeric compound that modulates expression of RNA in both single- and double-stranded forms. Multifunctional oligomeric compounds may be double-stranded oligomeric compounds or single-stranded oligomeric compounds comprising at least one double-stranded region.

As used herein, the term “optimized oligomeric compound” refers to an oligomeric compound which has properties balanced for maximum efficiency. Properties balanced include, but are not limited to, percent modulation of target RNA levels, propensity for cellular uptake, affinity for nucleic acid target and increased stability in the presence of nucleases. In some embodiments the oligomeric compound may be designed by balancing several factors, including, but not limited to, activity of the oligomeric compound, nuclease stability, location where inhibition is to be effected (nucleus v. cytoplasm), efficiency of delivery, ease of manufacturing, among others. For example, in some scenarios it may be desired to sacrifice some activity of the oligomeric compound in order to improve delivery of the oligomeric compound to its target.

As used herein, the term “optimized target region” refers to a target region that is hybridizable with an optimized oligomeric compound and/or is inhibitable both by ASO and RNAi oligomeric compounds and/or single- and double-stranded oligomeric compounds.

As used herein, the term “internal mismatch” refers to a mismatch within the core segment of an oligomeric compound. In some embodiments, an internal mismatch comprises no more than two, no more than four, no more that six, and no more than eight mismatched nucleobases.

As used herein, the term “external mismatch” refers to a mismatch within the 5′ segment or the 3′ segment of a nucleotide sequence. In some embodiments, an external mismatch comprises no more than two, no more than four, no more that six, and no more than eight mismatched nucleobases.

As used herein, the term “core segment” refers to nucleobases that fall between the 5′ segment and the 3′ segment of a nucleotide sequence. The 5′ segment comprises from about 2 to about 5 nucleobases at the 5′-termius of a nucleotide sequence while the 3′ segment comprises from about 2 to about 5 nucleobases at the 3′-termius of a nucleotide sequence.

In some embodiments the targeting process includes determination of at least one target region, segment, or site within the target nucleic acid for the antisense interaction to occur such that the desired effect, e.g., modulation of expression, will result.

As used herein the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

As used herein, the term “significant association” refers to a statistical association between variables (p<0.05). In some embodiments, a significant association refers to a statistical association between variables (p<0.01).

As used herein the term “tissue” refers to an aggregate of cells having a similar structure and function and includes constituent cells of the tissue. Constituent cells may include, without limitation, blood (e.g., hematopoietic cells (such as human hematopoietic progenitor cells, human hematopoietic stem cells, CD34⁺ cells CD4⁺ cells), lymphocytes and other blood lineage cells, bone marrow, brain, stem cells, blood vessel, liver, lung, bone, breast, cartilage, cervix, colon, cornea, embryonic, endometrium, endothelial, epithelial, esophagus, facia, fibroblast, follicular, ganglion cells, glial cells, goblet cells, kidney, lymph node, muscle, neuron, ovaries, pancreas, peripheral blood, prostate, skin, skin, small intestine, spleen, stomach, testes and fetal tissue.

Modifications and Linkages

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. In some embodiments, open linear structures are utilized. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Examples of oligomeric compounds useful in present invention include, but are not limited to, oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of the present specification, and as sometimes referenced in the art, modified oligonucleotides that do not have a phosphorus atom in their internucleoside backbone can also be considered to be oligonucleosides.

Exemplary modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. In some embodiments oligonucleotides having inverted polarity comprise a single 3′ to 3′ linkage at the 3′-most internucleotide linkage, i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Exemplary modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In some embodiments the present invention provides oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. The present invention also provides, in some embodiments, oligonucleotides having morpholino backbone structures of the above-referenced U.S. Pat. No. 5,034,506.

Modified oligonucleotides may also contain one or more substituted sugar moieties. In some embodiments oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments the 2′ position comprises O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other exemplary oligonucleotides comprise one or more of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments the modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification provided by the present invention includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

Other modifications include, but are not limited to, 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In some embodiments the 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278). In some embodiments 5-methylcytosine substitutions are combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an anti-diabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes oligomeric compounds which are chimeric compounds. “Chimeric” oligomeric compounds or “chimeras,” in the context of this invention, are oligomeric compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

In some embodiments the oligomeric compounds are synthesized in vitro and do not include antisense compositions of biological origin, or genetic vector constructs designed to direct the in vivo synthesis of oligomeric molecules.

In some embodiments, the present invention provides oligomeric compounds designed to target a non-structured region in an RNA target. In some embodiments, the oligomeric compounds have mismatches with the target region. In some embodiments the mismatches are external or internal mismatches. In some embodiments, the mismatch is a 2, 4, 6, or 8 base internal or 2, 4, 6, or 8 base external mismatch. In some embodiments, the oligomeric compounds have at least 75%, at least 80%, at least 85%, at least 90%, at least 95%, at least 98%, and at least 99% homology to the complement of the target region.

In some embodiments, the oligomeric compounds have alternating linkages and/or modifications.

As used herein, the term “alternating” is used herein to refer to every other one of a series. For example, in the context of nucleotide linkages, every other nucleotide may be linked by a phosphorothioate linkage while the remaining linkages are phosphodiester. Similarly, in the context of “alternating modifications”, in some embodiments every other nucleotide may have a 2′ modification.

In some embodiments, the oligomeric compounds have linkages and/or modifications that repeat in a consistent manner. For example, in the context of nucleotide linkages, every third nucleotide may be linked by a phosphorothioate linkage while the remaining linkages are phosphodiester.

In some embodiments, the oligomeric compounds may have blocks of modifications or modified linkages. For example, in some embodiments, 2, 3, 4, 5, 6, 7, 8, 9, 10 or more consecutive nucleotides have may a 2′ modification. In some embodiments, 2, 3, 4, 5, 6, 7, 8, 9, 10 or more consecutive nucleotides have may a modified linkage.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Target Regions

In some embodiments the oligomeric compounds of the present invention are targeted to or not targeted to one or more regions of the target nucleobase sequence. For example, in the context of a target nucleobase sequence comprising 5000 nucleobases, the oligomeric compounds are targeted to or are not targeted to regions comprising nucleobases 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, 2001-2050, 2051-2100, 2101-2150, 2151-2200, 2201-2250, 2251-2300, 2301-2350, 2351-2400, 2401-2450, 2451-2500, 2501-2550, 2551-2600, 2601-2650, 2651-2700, 2701-2750, 2751-2800, 2801-2850, 2851-2900, 2901-2950, 2951-3000, 3001-3050, 3051-3100, 3101-3150, 3151-3200, 3201-3250, 3251-3300, 3301-3350, 3351-3400, 3401-3450, 3451-3500, 3501-3550, 3551-3600, 3601-3650, 3751-3700, 3701-3750, 3751-3800, 3801-3850, 3851-3900, 3901-3950, 3951-4000, 4001-4050, 4051-4100, 4101-4150, 4151-4200, 4201-4250, 4251-4300, 4301-4350, 4351-4400, 4401-4450, 4451-4500, 4501-4550, 4551-4600, 4601-4650, 4751-4700, 4701-4750, 4751-4800, 4801-4850, 4851-4900, 4901-4950, or 4951-5000, or any combination or subcombination thereof. In some embodiments, the oligomeric compounds are targeted or are not targeted to one or more regions of the target nucleobase sequence comprising the 5′ UTR, the start region, the coding region, the stop region, or 3′ UTR, or any combination or subcombination thereof. In some embodiments the oligomeric compounds are targeted to the 3′UTR.

In some embodiments, the target segments of the present invention may also be combined with their respective complementary oligomeric compounds to form stabilized double-stranded (duplexed) oligonucleotides.

In some embodiments, the target region is localized to CoRest, Notch (Drosophila) homolog 2, PAK1, caspase recruitment domain 4, or glycogen synthase kinase 3 alpha, PTEN, CD54, ICAM and the like.

In some embodiments, oligomeric compounds are designed to target regions of nucleic acids having secondary structure. In some embodiments, nucleic acids having secondary structure which correspond to the structure descriptor elements are identified by searching at least one database. Structure descriptor elements may be determined as described in U.S. Ser. No. 09/076,440, filed May 12, 1998, and in U.S. Ser. No. 09/200,355, filed Nov. 25, 1998, each of which is incorporated by reference in its entirety. Any genetic database can be searched. In some embodiments the database is a UTR database, a compilation of the untranslated regions in messenger RNAs. A UTR database is accessible through the Internet at, for example, ftp://area.ba.cnr.it/pub/embnet/database/utr/. In some embodiments the database is searched using a computer program, such as, for example, RNAMOT, a UNIX-based motif searching tool available from Daniel Gautheret. Each “new” sequence that has the same motif is then queried against public domain databases to identify additional sequences. Results are analyzed for recurrence of pattern in UTRs of these additional ortholog sequences, as described below, and a database of RNA secondary structures is built. One skilled in the art is familiar with RNAMOT. Briefly, RNAMOT takes a descriptor string and searches any Fasta format database for possible matches. Descriptors can be very specific, to match exact nucleotide(s), or can have built-in degeneracy. Lengths of the stem and loop can also be specified. Single stranded loop regions can have a variable length. G-U pairings are allowed and can be specified as a wobble parameter. Allowable mismatches can also be included in the descriptor definition. Functional significance is assigned to the motifs if their biological role is known based on previous analysis. Known regulatory regions such as Iron Response Element have been found using this technique. In embodiments of the invention in which a database containing prokaryotic molecular interaction sites is compiled, in some embodiments human sequences are not searched or, alternatively, human sequences are discarded when found.

In some embodiments, the nucleic acids identified by searching databases such as, for example, searching a UTR database using Rnamot, are clustered and analyzed so as to determine their location within the genome. The results provided by RNAMOT identify sequences containing the secondary structure but do not give any indication as to the location of the sequence in the genome. Clustering and analysis is may then be performed with ClustalW, as described above, or with other commercially available products known to the art skilled.

In some embodiments of the invention, after clustering and analysis is performed, orthologs are identified as described above. However, in contrast to the orthologs identified above, which were, in some embodiments, identified on the basis of their primary nucleotide sequences, these new orthologous sequences may be identified on the basis of structure using the nucleic acids identified using RNAMOT. In some embodiments identification of orthologs is performed by BlastParse or Q-Compare, as described above. In embodiments of the invention in which a database containing prokaryotic molecular interaction sites is compiled, in some embodiments human orthologs are not searched or, alternatively, human orthologs are discarded when found.

After nucleic acids having secondary structures which correspond to the structure descriptor elements are identified, any or all of the nucleotide sequences can be compiled into a database by standard compiling protocols known to those skilled in the art. One database may contain eukaryotic molecule interaction sites and another database may contain prokaryotic molecule interaction sites.

Modulation of Expression

In some embodiments, modulation of RNA expression is inhibition (decrease) in RNA expression. Modulation of RNA expression may be determined by measuring RNA levels.

In some embodiments, RNA expression is inhibited at least 30%, at least 50%, at least 60%, least 70%, least 75%, least 80%, at least 85%, at least 90%, at least 95%, least 99%, and 100%, all as compared to a control.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

Data Analysis

Analysis of data relating to oligomeric compounds and/or target regions can be performed by methods well known to the art skilled. In some embodiments, data analysis involve one or more of Correlation analyses (Pearson's r, Spearmans rho, Spearman's rank, for example), regression analyses, Sensitivity analyses, Specificity analyses, and ROC analyses, among others.

In some embodiments ROC analysis is utilized to compare ASO and siRNA values and yields an area under the curve of at least 0.5, at least 0.6, at least 0.7, at least 0.8, or at least 0.9.

All references, Genbank accessions, patents and patent applications cited herein are herein incorporated by reference in their entirety.

As those skilled in the art will appreciate, numerous changes and modifications may be made to the exemplified embodiments of the invention without departing from the spirit of the invention. It is intended that all such variations fall within the scope of the invention.

### EXAMPLES

### Example 1

### Materials and Methods

Oligonucleotide Synthesis

Synthesis and purification of phosphorothioate modified oligodeoxynucleotides or chimeric 2′-O-methoxy-ethyl/deoxy phosphorothioate modified oligonucleotides was performed using an Applied Biosystems 380B automated DNA synthesizer as previously (McKay, R. A., et al. (1999) J Biol Chem, 274(3), 1715-22; Baker, B. F., et al. (1997), Journal Of Biological Chemistry, 272(18), 11994-2000). Sequences of oligonucleotides and placement of 2′-O-methoxy-ethyl modifications are detailed in Tables I and II.

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester-protecting group on the 2′-hydroxyl. This set of protecting groups is then used with standard solid-phase synthesis technology. It is, important to lastly remove the acid labile orthoester-protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester-protecting group developed by Dharmacon Research (Lafayette, Colo.), is one example of a useful orthoester-protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862: Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Boulder, CO). Once synthesized, complementary RNA antisense compounds can then be annealed by methods known in the art to form double-stranded (duplexed) antisense compounds. For example, duplexes can be formed by combining 30 μl of each of the complementary strands of RNA oligonucleotides (50 μM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

Table I. Sequence of CD54 RNase H-dependent oligonucleotides and siRNAs. All oligonucleotides are full phosphorothioate with 2′-O-methoxyethyl substitutions at positions 1-6 and 15-20 (bold). Residues 7-14 are unmodified 2′-deoxy so they can serve as substrates for RNaseH. The corresponding siRNAs use the same start but are 19 rather than 20 nucleotides in length and have dTdT additions at the 3′ end of each strand. Genbank accession # for CD54: J03132

| | | | | |
|---|---|---|---|---|
| SEQ ID | | START | | |
| NO: | ISIS # | POSITION | SEQUENCE | REGION |

| | | | | |
|---|---|---|---|---|
| 1 | 121725 | 8 | AGAGGAGCTCAGCGTCGACT | 5′UTR |
| 2 | 121726 | 33 | GGCTGAGGTTGCAACTCTGA | 5′UTR |
| 3 | 121727 | 256 | CCAGGCAGGAGCAACTCCTT | Coding |
| 4 | 121728 | 321 | TTGAATAGCACATTGGTTGG | Coding |
| 5 | 121729 | 422 | GCCCACTGGCTGCCAAGAGG | Coding |
| 6 | 121730 | 571 | TCTCTCCTCACCAGCACCGT | Coding |
| 7 | 121731 | 674 | AAAGGTCTGGAGCTGGTAGG | Coding |
| 8 | 121732 | 732 | GCGTGTCCACCTCTAGGACC | Coding |
| 9 | 121733 | 801 | CCAGTGCCAGGTGGACCTGG | Coding |
| 10 | 121734 | 921 | CCAGTATTACTGCACACGTC | Coding |
| 11 | 121735 | 1002 | CCTCTGGCTTCGTCAGAATC | Coding |
| 12 | 121736 | 1121 | GGTGGCCTTCAGCAGGAGCT | Coding |
| 13 | 121737 | 1221 | CATACAGGACACGAAGCTCC | Coding |
| 14 | 121738 | 1341 | CATCCTTTAGACACTTGAGC | Coding |
| 15 | 121739 | 1421 | GCTCCTGGCCCGACAGAGGT | Coding |
| 16 | 121740 | 1501 | GCTACCACAGTGATGATGAC | Coding |
| 17 | 121741 | 1622 | TTGTGTGTTCGGTTTCATGG | Coding |
| 18 | 121742 | 1633 | GGAGGCGTGGCTTGTGTGTT | Coding |
| 19 | 121743 | 1654 | CCTGTCCCGGGATAGGTTCA | Coding |
| 20 | 121744 | 1666 | CGAGGAAGAGGCCCTGTCCC | 3′UTR |
| 21 | 121745 | 1711 | TCCACTCTGTTCAGTGTGGC | 3′UTR |
| 22 | 121746 | 1781 | TCTGACTGAGGACAATGCCC | 3′UTR |
| 23 | 121747 | 1818 | TAGGTGTGCAGGTACCATGG | 3′UTR |
| 24 | 121748 | 1924 | CCTCTCATCAGGCTAGACTT | 3′UTR |
| 25 | 121749 | 1971 | CCAGTTGTATGTCCTCATGG | 3′UTR |
| 26 | 121750 | 2012 | GGGCCTCAGCATACCCAATA | 3′UTR |
| 27 | 121751 | 2056 | ATGCTACACATGTCTATGGA | 3′UTR |
| 28 | 121752 | 2100 | GCCCAAGCTGGCATCCGTCA | 3′UTR |
| 29 | 121753 | 2103 | AGTGCCCAAGCTGGCATCCG | 3′UTR |
| 30 | 121754 | 2221 | GCTCCGTGAGGCCAGAGACC | 3′UTR |
| 31 | 121755 | 2291 | CAGGCACTCTCCTGCAGTGT | 3′UTR |
| 32 | 121756 | 2341 | GAAAGGCAGGTTGGCCAATG | 3′UTR |
| 33 | 121757 | 2417 | GGTAATCTCTGAACCTGTGA | 3′UTR |
| 34 | 121758 | 2531 | GTCCAGACATGACCGCTGAG | 3′UTR |
| 35 | 121759 | 2619 | CTGGAGCTGCAATAGTGCAA | 3′UTR |
| 36 | 121760 | 2731 | TACACATACACACACACACA | 3′UTR |
| 37 | 121761 | 2831 | GCTGAGGTGGGAGGATCACT | 3′UTR |
| 38 | 121762 | 2871 | GGTGTGGTGTTGTGAGCCTA | 3′UTR |
| 39 | 121763 | 2944 | CTAACACAAAGGAAGTCTGG | 3′UTR |
| 40 | 121764 | 3104 | CAGTGCCCAAGCTGGCATCC | 3′UTR |

Table II. Sequence of human PTEN RNase H-dependent oligonucleotides and siRNAs. All oligonucleotides are full phosphorothioate with 2′-O-methoxyethyl substitutions at positions 1-4 and 15-18 (bold). Residues 5-14 are unmodified 2′-deoxy so they can serve as substrates for RNaseH. The corresponding siRNAs use the same start position, but are 19 rather than 18 nucleotides in length and have dTdT additions at the 3′ end of each strand. Genbank accession # for PTEN: U92436

| | | | | |
|---|---|---|---|---|
| SEQ ID | | START | | |
| NO. | ISIS # | POSITION | SEQUENCE | REGION |

| | | | | |
|---|---|---|---|---|
| 41 | 29574 | 19 | CGAGAGGCGGACGGGACC | 5′UTR |
| 42 | 29575 | 57 | CGGGCGCCTCGGAAGACC | 5′UTR |
| 43 | 29576 | 197 | TGGCTGCAGCTTCCGAGA | 5′UTR |
| 44 | 29577 | 314 | CCCGCGGCTGCTCACAGG | 5′UTR |
| 45 | 29578 | 421 | CAGGAGAAGCCGAGGAAG | 5′UTR |
| 46 | 29579 | 494 | GGGAGGTGCCGCCGCCGC | 5′UTR |
| 47 | 29581 | 671 | CCGGGTCCCTGGATGTGC | 5′UTR |
| 48 | 29582 | 757 | CCTCCGAACGGCTGCCTC | 5′UTR |
| 49 | 29583 | 817 | TCTCCTCAGCAGCCAGAG | 5′UTR |
| 50 | 29584 | 891 | CGCTTGGCTCTGGACCGC | 5′UTR |
| 51 | 29585 | 952 | TCTTCTGCAGGATGGAAA | 5′UTR |
| 52 | 29587 | 1106 | GGATAAATATAGGTCAAG | Coding |
| 53 | 29588 | 1169 | TCAATATTGTTCCTGTAT | Coding |
| 54 | 29589 | 1262 | TTAAATTTGGCGGTGTCA | Coding |
| 55 | 29590 | 1342 | CAAGATCTTCACAAAAGG | Coding |
| 56 | 29591 | 1418 | ATTACACCAGTTCGTCCC | Coding |
| 57 | 29592 | 1504 | TGTCTCTGGTCCTTACTT | Coding |
| 58 | 29593 | 1541 | ACATAGCGCCTCTGACTG | Coding |
| 59 | 29595 | 1694 | GAATATATCTTCACCTTT | Coding |
| 60 | 29596 | 1792 | GGAAGAACTCTACTTTGA | Coding |
| 61 | 29597 | 1855 | TGAAGAATGTATTTACCC | Coding |
| 62 | 29599 | 2020 | GGTTGGCTTTGTCTTTAT | Coding |
| 63 | 29600 | 2098 | TGCTAGCCTCTGGATTTG | Coding |
| 64 | 29601 | 2180 | TCTGGATCAGAGTCAGTG | Coding |
| 65 | 29602 | 2268 | TATTTTCATGGTGTTTTA | 3′UTR |
| 66 | 29603 | 2347 | TGTTCCTATAACTGGTAA | 3′UTR |
| 67 | 29604 | 2403 | GTGTCAAAACCCTGTGGA | 3′UTR |
| 68 | 29605 | 2523 | ACTGGAATAAAACGGGAA | 3′UTR |
| 69 | 29606 | 2598 | ACTTCAGTTGGTGACAGA | 3′UTR |
| 70 | 29607 | 2703 | TAGCAAAACCTTTCGGAA | 3′UTR |
| 71 | 29608 | 2765 | AATTATTTCCTTTCTGAG | 3′UTR |
| 72 | 29609 | 2806 | TAAATAGCTGGAGATGGT | 3′UTR |
| 73 | 29610 | 2844 | CAGATTAATAACTGTAGC | 3′UTR |
| 74 | 29611 | 2950 | CCCCAATACAGATTCACT | 3′UTR |
| 75 | 29612 | 3037 | ATTGTTGCTGTGTTTCTT | 3′UTR |
| 76 | 29613 | 3088 | TGTTTCAAGCCCATTCTT | 3′UTR |

TABLE III
Sequences of ASOs targeting intronic sequence

| SEQ ID | | | | |
|---|---|---|---|---|
| NO: | GENE NAME | ISIS # | SEQUENCE | location |
| 77 | CoRest | 165031 | AATCCCAGCTACTCGGGAGG | intron 2 |
| 78 | Notch | 226968 | AAGCCCTTACTTGCATGTCT | exon 25:intron 25 |
| | (Drosophila) | | | |
| | homolog 2 | | | |
| 79 | PAK1 | 232214 | GCCTGAAGCACTGAACAGTA | intron 5 |
| 80 | caspase | 199213 | CGAGCTATTACCACAGTATT | exon 11:intron 11 |
| | recruitment | | | |
| | domain 4 | | | |
| 81 | glycogen | 116648 | AGCCAATGACACCATACCTT | intron 1 |
| | synthase | | | |
| | kinase 3 | | | |
| | alpha | | | |

Cell Culture

T24 Cells:

T24 cells (American Type Tissue Culture Collection, Rockville, Md.) were cultivated in DMEM supplemented with 10% fetal bovine serum in 6 well culture dishes at a density of 250,00 cells/well. Cells were treated with oligonucleotides as described previously (Chiang, M.-Y., et al. (1991) J. Biol. Chem., 266(27), 18162-18171; Vickers et al. (2000) Nucleic Acids Res., 28(6), 1340-1347). For RNase H-dependent antisense oligonucleotides, cells were incubated with a mixture of 3 μg/ml LIPOFECTIN™ Reagent (transfection reagent; Invitrogen, Carlsbad, Calif.) per 100 nM oligonucleotide in OPTIMEM® growth and maintenance media (Invitrogen, Carlsbad, Calif.). The LIPOFECTIN™ Reagent (transfection reagent; Invitrogen, Carlsbad, Calif.) concentration used with siRNAs was 6 μg/ml per 100 nM RNA duplex. Concentrations reported herein represent concentration of the duplex. After 4 hours the transfection mixture was aspirated from the cells and replaced with fresh DMEM plus 10% FCS and incubated at 37° C., 5% CO₂ until harvest.

To induce CD54 mRNA expression, oligonucleotide treated cells were incubated overnight then treated with 5 ng/ml TNF-α (R&D Systems, Minneapolis, Minn.) for 2-3 hours prior to harvest of cells for RNA expression analysis. For analysis of cell surface expression of CD54 protein, cells were induced with 5 ng/ml TNF-α immediately following the transfection, and incubated overnight.

Primary Mouse Hepatocytes:

Primary mouse hepatocytes were prepared from CD-1 mice purchased from Charles River Labs. Primary mouse hepatocytes were routinely cultured in Hepatocyte Attachment Media (Invitrogen Life Technologies, Carlsbad, Calif.) supplemented with 10% Fetal Bovine Serum (Invitrogen Life Technologies, Carlsbad, Calif.), 250 nM dexamethasone (Sigma-Aldrich Corporation, St. Louis, Mo.), 10 nM bovine insulin (Sigma-Aldrich Corporation, St. Louis, Mo.).

Cells were seeded into 96-well plates (Falcon-Primaria #353872, BD Biosciences, Bedford, Mass.) at a density of 4000-6000 cells/well for use in antisense oligonucleotide transfection. For cells grown in 96-well plates, cells were treated with 100 μL of OPTI-MEM-1 containing 2.5 μg/mL LIPOFECTIN (Invitrogen Corporation, Carlsbad, Calif.) and the desired concentration of oligonucleotide. Cells were treated and data obtained in triplicate. After 4 hours of treatment at 37° C., the medium was replaced with fresh medium. Cells were harvested 16-24 hours after oligonucleotide treatment.

For Northern blotting or other analyses, cells may be seeded onto 100 mm or other standard tissue culture plates and treated similarly, using appropriate volumes of medium and oligonucleotide.

### Example 2

### Real-Time Quantitative PCR Analysis of mRNA Levels

Total RNA was harvested at the indicated times following the beginning of transfection using an RNeasy Mini prep kit (Qiagen, Valencia, Calif.) according to the manufacturers protocol. Gene expression was analyzed using quantitative RT/PCR essentially as described (Winer, J., et al. (1999) Development and Validation of Real-Time Quantitative Reverse Transcriptase±Polymerase Chain Reaction for Monitoring Gene Expression in Cardiac Myocytes in Vitro. Analytical Biochemistry, 270,41-49). This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Briefly, 200 ng of Total RNA was analyzed in a final volume of 50 μl containing 200 nM gene specific PCR primers, 0.2 mM of each dNTP, 75 nM fluorescently labeled oligonucleotide probe, 1 X RT/PCR buffer, 5 mM MgCl_{2,} 2U Platinum® Taq DNA Polymerase (Invitrogen, Carlsbad, Calif.), and 8U ribonuclease inhibitor. Reverse transcription was performed for 30 minutes at 48° C. followed by PCR: 40 thermal cycles of 30 s at 94° C. and 1 minute at 60° C. using an ABI PRISM®7700 Sequence Detector (Foster City, Calif.).

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

The following primer/probe sets were used.

C-raf kinase (accession number X03484):

| | | |
|---|---|---|
| forward primer- | AGCTTGGAAGACGATCAGCAA, | (SEQ ID NO:82) |
| reverse primer- | AAACTGCTGAACTATTGTAGGAGAGATG, | (SEQ ID NO:83) |
| probe- | AGATGCCGTGTTTGATGGCTCCAGC. | (SEQ ID NO:84) |

CD54 (accession number J03132):

| | | |
|---|---|---|
| forward primer- | CATAGAGACCCCGTTGCCTAAA, | (SEQ ID NO:85) |
| reverse primer- | TGGCTATCTTCTTGCACATTGC, | (SEQ ID NO:86) |
| probe- | CTCCTGCCTGGGAACAACCGGAA. | (SEQ ID NO:87) |

PTEN (accession number U92436):

| | | |
|---|---|---|
| forward primer- | AATGGCTAAGTGAAGATGACAATCAT, | (SEQ ID NO:88) |
| reverse primer- | TGCACATATCATTACACCAGTTCGT, | (SEQ ID NO:89) |
| probe- | TTGCAGCAATTCACTGTAAAGCTGGAAAGG. | (SEQ ID NO:90) |

Bel-x (accession number Z23115):

| | | |
|---|---|---|
| forward primer- | TGCAGGTATTGGTGAGTCGG, | (SEQ ID NO:91) |
| reverse primer- | TCCAAGGCTCTAGGTGGTCATT, | (SEQ ID NO:92) |
| probe- | TCGCAGCTTGGATGGCCACTTACCT. | (SEQ ID NO:93) |

G3PDH (accession number X01677):

| | | |
|---|---|---|
| forward | GAAGGTGAAGGTCGGAGTC, | (SEQ ID NO:94) |
| primer- | | |
| reverse | GAAGATGGTGATGGGATTTC, | (SEQ ID NO:95) |
| primer- | | |
| probe- | CAAGCTTCCCGTTCTCAGCC. | (SEQ ID NO:96) |

COREST (accession number NM_{—}015156):

| | | |
|---|---|---|
| forward primer- | ACAATCCCATTGACATTGAGGTT, | (SEQ ID NO:97) |
| reverse primer- | TTTGCTCTATTTTTAGCTTGTGTGCT, | (SEQ ID NO:98) |
| probe- | AAGGAGGTTCCCCCTACTGAGACAGTTCCT. | (SEQ ID NO:99) |

Notch homolog 2 (accession number NM_{—}024408):

| | | |
|---|---|---|
| forward primer- | TGGCAACTAACGTAGAAACTCAACA, | (SEQ ID NO:100) |
| reverse primer- | TGCCAAGAGCATGAATACAGAGA, | (SEQ ID NO:101) |
| probe- | ACAACTATAGACTTGCTCATTGTTCAGACTGATTGCC. | (SEQ ID NO:102) |

PAK1 (accession number U51120):

| | | |
|---|---|---|
| forward primer- | TGTGATTGAACCACTTCCTGTCA, | (SEQ ID NO:103) |
| reverse primer- | GGAGTGGTGTTATTTTCAGTAGGTGAA, | (SEQ ID NO:104) |
| probe- | TCCAACTCGGGACGTGGCTACA. | (SEQ ID NO:105) |

CARD-4 (accession number NM_{—}006092):

| | | |
|---|---|---|
| forward primer- | GCAGGCGGGACTATCAGGA, | (SEQ ID NO:106) |
| reverse primer- | AGTTTGCCGACCAGACCTTCT, | (SEQ ID NO:107) |
| probe- | TCCACTGCCTCCATGATGCAAGCC. | (SEQ ID NO:108) |

### Example 3

### Assays

### Flow Cytometry

Following oligonucleotide treatment, cells were detached from the plates with Dulbecco's phosphate buffered saline (D-PBS) (without calcium and magnesium) supplemented with 4 mM EDTA. Cells were transferred to microcentrifuge tubes, pelleted at 5000 rpm for 1 minute and washed in 2% bovine serum albumin, 0.2% sodium azide in D-PBS at 4° C. PE anti-human CD54 antibody (Pharmingen #555511, San Diego, Calif.) was then added at 1:20 in 0.1 ml of the above buffer. The antibody was incubated with the cells for 30 minutes at 4° C. in the dark. Cells were washed again as above and resuspended in 0.3 ml of PBS buffer with 0.5% formaldehyde. Cells were analyzed on a Becton Dickinson FACScan. Results are expressed as percentage of control expression based upon the mean fluorescence intensity.

Luciferase Assays

Ten μg of plasmid pGL3-5132-S0 or pGL3-5132-S20 (Vickers et al. (2000) Nucleic Acids Res., 28(6), 1340-1347) was introduced into COS-7 cells at 70% confluency in a 10 cm dish using SUPERFECT® Reagent (transfection reagent; Qiagen). Following a 2 hour treatment, cells were trypsinized and split into a 24 well plate. Cells were allowed to adhere for 1 hour then ASO oligonucleotides or siRNA oligonucleotides were added in the presence of LIPOFECTIN™ Reagent (transfection reagent; Invitrogen, Carlsbad, Calif.) as detailed above. All oligonucleotide treatments were performed in duplicate or triplicate. Following the 4 hour oligonucleotide treatment, cells were washed and fresh DMEM+10% FCS was added. The cells were incubated overnight at 37° C. The following morning cells were harvested in 150 μl of Passive Lysis Buffer (Promega, Madison, Wis.). 60 μl of lysate was added to each well of a black 96 well plate followed by 50 μl Luciferase Assay Reagent (Promega). Luminescence was measured using a Packard TOPCOUNT™ (luminescence counter; Meriden, Conn.).

### Example 4

### Statistical Analyses of Gene Walk Data

Statistical analyses were conducted to examine the association between siRNA oligonucleotide and ASO oligonucleotide walks. Similarity between the two walks for a given gene was measured by using correlation coefficients and average distance. Two different correlation measures were employed: Pearson's product-moment correlation coefficient, which measures a linear relationship between siRNA and ASO walks, and Spearman's rank-order correlation coefficient, which measures a linear relationship between the potency of siRNA and ASO walks. One-sample one-tailed t-tests were conducted for observed correlation coefficients to assess whether they are significantly greater than the null hypothesis of no correlation. Statistical inference on observed average distance was conducted by randomizing sample pairs of siRNA and ASO walk. Again, one-tailed tests were used to determine whether the observed distances are significantly smaller than those expected from random chance. The association between siRNA and ASO walk was further examined by the receiver operating characteristic (ROC) analysis. Receiver operating characteristic (ROC) analysis is the standard approach to evaluate the sensitivity and specificity of diagnostic procedures (Swets et al., Evaluation of diagnostic systems: Methods from signal detection theory. Academic Press, New York, 1992). ROC analysis estimates a curve, which describes the inherent tradeoff between sensitivity and specificity of a diagnostic test. Each point on the ROC curve is associated with a specific diagnostic criterion. This point will vary among observers because their diagnostic criteria will vary even when their ROC curves are the same. The area under the ROC curve (A-z) has become a particularly important metric for evaluating diagnostic procedures because it is the average sensitivity over all possible specificities. (Hanley et al., Radiology 1982; 143:29-36).

siRNAs were classified as potent when the percent inhibition rate was smaller than the median value of 67.4% for the CD54 siRNA walk and 57.1% for PTEN walk. An arbitrary cutoff was then set for ASO walks. ASOs with percent inhibition rate smaller than this cutoff value were classified as potent. From the classification of siRNAs and ASOs, a 2-by-2 contingency table was constructed. Finally, true positive rate (TPR) and false positive rate (FPR) were determined based on this table. For example, TPR is the number of cases where potent ASOs correspond to potent siRNAs divided by the number of potent siRNAs. Similarly, FPR is the number of cases where potent ASOs corresponds to non-potent siRNAs divided by the number of non-potent siRNAs. For CD54, a cutoff value of 70% gives TPR=75% and FPR=45%. For the PTEN gene, a cutoff 40% gives TPR=72% and FPR=44%. By varying these cutoff values a ROC curve can be drawn on a plane spanned by FPR and TPR. The area under the ROC curve provides a measure of overall accuracy.

### Example 5

### Active RNase H-dependent Antisense Oligonucleotide Target Sites Predict siRNA Target Sites

As both siRNA and RNase dependent antisense oligonucleotides must first hybridize to target RNA and subsequently direct specific RNases to bind and cleave the bound RNA (Monia et al. (1993) Journal of Biological Chemistry, 268(19), 14514-22; Elbashir, S. M., et al. (2001), EMBO J, 20,6877-6888; Wu et al. (1999), J Biol Chem, 274(40), 28270-8), we examined whether an active RNase H dependent ASO site would also be an active siRNA site. Initially siRNAs were designed and synthesized based upon the target sequences of active ASOs previously identified. ISIS 5132 is a 20-base first generation phosphorothioate oligodeoxynucleotide that targets the 3′-untranslated region of human C-raf kinase mRNA and effectively and specifically reduces expression of both mRNA and protein (Monia et al. (1996), Proc Natl Acad Sci USA, 93(26), 15481-15484). An siRNA duplex (si5132) comprising 21-nt sense and 21-nt antisense strands was designed using the first 19 nucleotides of the target site for ISIS 5132 in the paired region and unpaired 2-nt 3′ dTdT overhangs. T24 cells were treated with either the parent ASO or with the siRNA at doses ranging from 3 to 300 nM as detailed in Examples. Total RNA was isolated from the cells the day following the transfection and levels of C-raf message determined using quantitative RT/PCR. Levels of G3PDH mRNA were also determined in order to normalize the data. Both ISIS 5132 and the corresponding siRNA to the same target site were found to inhibit the expression of the target, both with an IC₅₀ of approximately 50 nM. A siRNA targeted to a different gene had no effect on the expression of C-raf nor did a scrambled control version of ISIS 5132.

Chimeric oligonucleotides in which 2′-O-methoxyethyl substituted bases flank a central unmodified 2′-oligodeoxynucleotide region that serve as substrate for RNase H region have been shown to have increased potency as compared to phosphorothioate oligodeoxynucleotides (McKay et al. (1999), Journal of Biological Chemistry, 274(3), 1715-1722; Altmann et al. (1996), 50(4(April)), 168-176). ISIS 16009 is a 20 base chimeric oligonucleotide that has previously been demonstrated to be an effective inhibitor of human Bcl-X (Taylor, J. K., et al. (1999), Oncogene, 18(31), 4495-504). Another 20 base chimeric oligonucleotide, ISIS 116847, effectively inhibits expression of the human PTEN gene (Butler et al. (2002), Diabetes, 51(4), 1028-34). The siRNA versions, si 16009 and si 116847, as well as the homologous parent RNase H-dependant oligonucleotides were transfected into T24 cells at doses ranging from 10 to 200 nM. In both cases the second generation RNase H-dependent oligonucleotide was a slightly more potent inhibitor of mRNA expression than the corresponding siRNA. In the case of Bcl-X, the RNase H-dependent oligonucleotide has an IC₅₀ of approximately 30 nM, while the siRNA version, si16009, has an IC₅₀ of approximately 100 nM. PTEN is efficiently inhibited with IC₅₀s of 10 nM and 25 nM for the RNase H-dependent oligonucleotide and siRNA, respectively.

These results suggest that as for RNase H-dependent oligonucleotides [Chiang et al. (1991), J. Biol. Chem., 266(27), 18162-18171; Dean et al. (1994); Journal Of Biological Chemistry, 269(23), 16416-24; Monia et al. (1996), Nature Medicine, 2(6), 668-675; Eckardt et al. (1997), Biochemistry, 36(42), 12711-12721; Laptev (1994), Biochemistry, 33,11033-11039], not all sites on the target RNA are good target sites for siRNA molecules, as has recently been reported (Holen et al. (2002), Nucleic Acids Res., 30(8), 1757-1766). Since target accessibility cannot yet be accurately predicted based upon mRNA sequence, identification of potent antisense sequences is often based upon empirical approaches to oligonucleotide selection. Many investigators employ an oligonucleotide screen, in which multiple oligonucleotides are designed to hybridize to different regions on the target RNA and tested for direct inhibition of target gene expression, in order to identify potent antisense inhibitors (Dean et al. (1994), Journal Of Biological Chemistry, 269(23), 16416-24; Monia et al. (1996), Nature Medicine, 2(6), 668-675; Chiang et al. (1991), Journal of Biological Chemistry, 266(27), 18162-71; Goodchild et al. (1988), [published erratum appears in Proc Natl Acad Sci USA 1989 March; 86(5): 1504]. Proc Natl Acad Sci USA, 85,5507-5511; Cotter et al. (1994), Oncogene, 9, 3049-3055). To determine if the lack of activity of the CD54 siRNA molecules was due to suboptimal siRNA design or to blocking activity induced by TNF-α treatment, 40 siRNA and 40 second-generation chimeric oligonucleotides were designed to the same sites of the CD54 mRNA (Table 1). The siRNA duplexes comprised 21-nt sense and 21-nt antisense strands, paired in a manner to have a 19-nt duplex region and a 2-nt overhang at each 3′ terminus (Table I). The target sites included various regions of the human CD54 message including 5′-UTR (untranslated region), coding region and 3′UTR. T24 cells were treated with oligonucleotides at a single dose of 100 nM as described in the Examples. The results we determined as a percent of untreated control expression of induced CD54 message normalized to G3PDH mRNA expression in the same sample. Active sequences were identified in both the RNase H-dependent oligonucleotide and siRNA walks. In the RNase H-dependent oligonucleotide screen, 12 of 40 oligonucleotides were found to inhibit expression of CD54 mRNA by greater than 50% as compared to the untreated control, while the siRNA screen identified 9 of 40 sequences as active by the same criteria. Comparison of the active target sites revealed that 5 of the 9 active siRNA sites were also identified as active sites in the ASO screen. The data also indicates that there are regions of greater activity or “hot spots” for both siRNA and ASOs along the message. For example, homologous siRNAs and ASOs both show good activity in the approximately 200 nucleotide 3′-untranslated region from base 1781 to 1971. These results demonstrate that the lack of activity for the CD54 directed siRNA molecules is not due to induction of an inhibitory factor by TNF-α treatment.

Statistical analyses described above were applied to siRNA and ASO walk data for CD54 mRNA reduction. The data were comprised of two independent ASO screens and five independent siRNA screens that were averaged to produce composite siRNA/ASO walks. Pearson's correlation coefficient was determined to be 0.424 with p-value 0.0032 and Spearman's correlation coefficient was 0.426 with p-value 0.0039. The average distance between the two walks was 18.5% with p-value 0.0056. These results indicate that there is significant association between siRNA and ASO walks in terms of correlation coefficients and average distance. The association between siRNA and ASO walks was further analyzed using ROC analysis. The area under the ROC curve is a summary of the overall diagnostic accuracy of the test that measures the correspondence between potent siRNA and ASO sites. The area under the ROC curve is 0.75 for CD54.

Cell surface CD54 protein expression was also evaluated by flow cytometry. Comparison of mRNA reduction and protein reduction for the siRNA walk was performed. In general the results are highly correlated with the same active targets identified by either mRNA or protein reduction. However, the contrast between the siRNA and ASO screens appears to be more striking when evaluated at the protein level. In this assay 23 of the 40 ASOs were identified as active while 17 of the 40 siRNAs met the same criteria for activity. Comparison of the active sequences revealed that 11 of the 17 active siRNAs were common with actives in the ASO walk.

A second comparative screen was performed using 36 second generation chimeric oligonucleotides, 18 nucleotides in length, and a series of corresponding siRNA duplexes (Table II) targeted to the human PTEN message. PTEN is constitutively expressed in T24 cells. Cells were treated with siRNAs or ASOs as described supra. As defined by a target mRNA reduction of 50% or greater, 22 of the 36 ASOs were identified as active. The siRNA walk identified 12 of 36 sites as active as defined by the same criteria. However, of these 12 active sites, 10 were shared as actives with the ASO screen, with only 2 of the active siRNAs not identified in the ASO screen.

The ASO/siRNA screens for PTEN were repeated 3 separate times. A statistical analysis of the composite data from the three experiments was performed as detailed above. Pearson's correlation coefficient was determined to be 0.425 with p-value 0.0049 and Spearman's correlation coefficient was 0.318 with p-value 0.0299. The average distance between the two walks was 21.3% with p-value 0.0038. These results suggest that there is significant association between siRNA and ASO walks in terms of Pearson's correlation coefficient and average distance. ROC analysis of this data gives a value 0.588 for PTEN. As a result of gene walk data, there is a reasonable correlation between siRNA and ASO active sites for PTEN.

### Example 6

### Modulation of Inducible Genes

The activities of ASOs and siRNAs were also compared in an inducible gene system. ISIS 2302, a first generation phosphorothioate oligodeoxynucleotide that hybridizes to the 3′-untranslated region of human CD54 (ICAM-1), was previously shown to be a potent and specific inhibitor of CD54 expression (Bennett et al. (1994), J Immunol, 152(7), 3530-40). ISIS 2302 or the siRNA targeting the same sequence, si2302, was administered to T24 cells in the presence of LIPOFECTIN™ Reagent (transfection reagent; Invitrogen, Carlsbad, Calif.) at a dose of 200 nM for four hours. Cells were then incubated overnight and the following day CD54 mRNA expression was induced by treating the cells with 5 ng/ml of TNF-α for 2 hours. Total RNA was harvested and ICAM mRNA expression was analyzed by qRT/PCR. While ISIS 2302 reduced inducible ICAM-1 expression by 85%, si2302 appeared to have no inhibitory effect on message levels (data not shown). Recently Tushl and co-workers reported a simple method for design of active siRNA duplexes (Elbashir et al. (2002), Methods, 26(2), 199-213). We have designed two siRNAs targeting CD54 based upon this method. The target was searched for the sequences 5′-AA(N₁₉)-3′, where N is any nucleotide, in the mRNA sequence. Two oligonucleotides were identified that meet these criteria; 170 nucleotides and 224 nucleotides from the AUG translation codon, respectively. Neither of these siRNAs (generated according to the Tuschl method) appeared to reduce the expression of the targeted message.

As discussed above, ICAM-1 expression was induced with TNF-α. The siRNA molecules designed to hybridize to over 40 distinct sites on the ICAM-1 mRNA resulted in several siRNA molecules that effectively reduced ICAM-1 expression and, in general, activity correlated with the activity to RNase H-dependent oligonucleotides designed to the same site.

### Example 7

### Effect of RNA Secondary Structure on Activity

The secondary structure of the mRNA target influences activity of ASOs in cell culture (Vickers et al. (2000), Nucleic Acids Res., 28(6), 1340-1347). A luciferase reporter system was developed in which the target site for ISIS 5132 was cloned into the 5′UTR of the luciferase reporter plasmid pGL3-Control. Sequence immediately adjacent to the target sequence was altered to form various RNA secondary structures that included the 5132 sequence. These structures ranged from one in which the entire target site was sequestered in a 20 base stem closed by a UUGC tetraloop (pGL3-5132-S20) to one that had little predicted secondary structure likely to inhibit hybridization of ASO to target (pGL3-5132-S0).

The activity of ISIS 5132 and si5132 were compared using the pGL3-5132-S20 and pGL3-5132-S0 constructs. The reporter plasmids were transfected into COS-7 cells as detailed in above. Following the plasmid transfection, cells were seeded in 24-well plates and treated with ISIS 5132 or si5132 at doses ranging from 10 to 300 nM. Lysates from the treated cells were assayed for luciferase activity 16 hours later. When directed against the message with no structure (pGL3-5132-S0), both ISIS 5132 and si5132 effectively reduced luciferase expression in a dose dependant manner with IC₅₀s between 30 and 100 nM, which is consistent with the observed IC₅₀ for endogenous message reduction. Neither the ASO nor siRNA was found to inhibit luciferase (a non-endogenous, reporter gene) expression when directed against the highly structured target (pGL3-5132-S20). Even at the highest dose tested (300 nM) there was no inhibition observed. The secondary structure of the target appears to have an effect on the antisense mechanisms of siRNAs and ASOs.

### Example 8

### Sequence Specificity of RNase H-dependent Oligonucleotides and siRNA

The sequence fidelity of the RNAi pathway has been evaluated to a limited extent in several hallmark systems, including C. elegans (Parrish et al. (2000), Molecular Cell, 6,1077-87) and Drosophila cell extracts (Elbashir et al. (2001), EMBO J, 20,6877-6888), and most recently in mammalian cell culture (Elbashir et al. (2001), Nature, 411(6836), 494-8; Holen et al. (2002). Nucleic Acids Res, 30(8), 1757-1766). Several investigators have reported that incorporation of one or two mismatches into a siRNA construct, with respect to the target RNA, is sufficient to disable RNAi activity against the target RNA. A common attribute of each of the mismatch constructs tested thus far however, has been the location of the mismatches in the center domain of the construct. To further define the fidelity of the RNAi pathway for perfect Watson-Crick base pair matched sequences, we tested an additional type of construct, wherein a mismatch was incorporated in each of the 5′ and 3′ terminal domains of the siRNA targeting PTEN (si116847). The same mismatches were also incorporated into the ASO, ISIS 116847. When the mismatches were placed in the center of the sequence there was a complete loss of activity for both RNase H-dependent oligonucleotide and siRNA. In contrast to the duplex with two mismatches positioned in the center of the siRNA, the siRNA with mismatches in the outside domains demonstrated only a moderate loss of activity in comparison to the perfect match construct. The results for the ASO were very similar, although the RNase H-dependent oligonucleotide containing mismatches on the ends demonstrated a greater loss of activity than was observed for the homologous siRNA (71% vs. 52% control).

### Example 9

### Comparison of Potency and Efficacy

Comparison of the relative potency of siRNAs directed to the same site on the target RNA as an optimized RNase H-dependent oligonucleotide revealed that the RNase H oligonucleotide exhibited similar or better activity to the siRNA. The siRNA and RNase H-dependent oligonucleotides also exhibited a similar level of efficacy. Since the siRNA molecules used for these analysis were not selected as the optimal siRNA molecules for the respective target based upon screening numerous siRNA sequences, we compared the most effective siRNA molecule derived from the siRNA screen with an optimized second-generation chimeric oligonucleotide to PTEN. The different antisense agents, tested at concentrations ranging from 10 nM to 200 nM in T24 cells, produced a similar dose-response curve with an IC₅₀ value near 10 rM. Additionally, both agents maximally reduced PTEN expression by greater than 90%.

Similarly, the most effective siRNA from the CD54 screen was compared with its corresponding second-generation chimeric oligonucleotide, which showed a similar degree of efficacy in the primary screen. The siRNA si121747 or the oligonucleotide ISIS 121747 was administered to T24 cells at doses ranging from 10 to 200 nM. Following induction of CD54 message by TNF-alpha treatment mRNA reduction was accessed by qRT/PCR. As with PTEN, siRNA and oligonucleotide produced similar dose-response curves with IC₅₀ values of approximately 15 nM for the siRNA and 30 nM for the oligonucleotide. The efficacy was almost identical with maximal reduction of approximately 85% for both antisense agents.

### Example 10

### Duration of Action

Experiments in plants (Vance, (2001), Science, 292,2277-2280; Waterhouse et al. (2001), Nature, 411,834-842) and nematodes (Grishok et al. (2000), Science, 287(5462), 2494-7) have suggested the existence of a system whereby certain siRNA genes are involved in the heritability of RNAi-induced phenotypes. However, it has recently been proposed that gene expression recovers within 4-5 days of transfection of siRNAs in human cells suggesting against the presence of a propagative system in human cells for siRNA molecules (Holen, T., et al. (2002), Nucleic Acids Res, 30(8), 1757-1766). Antisense activity has previously been shown to persist in cell culture from 3-7 days, depending upon cell type, culture conditions, and type of chemistry. The duration of action of a second generation RNase H dependent oligonucleotide was compared to siRNA activity in T24 cells using human Bcl-X as a target. Cells were seeded in 6 well dishes so that they would be 80-90% confluent at the time of harvest. Oligonucleotide treatment was at 100 nM with ISIS 16009 or si16009 as detailed above. Total RNA was harvested 8, 24, 48, 72, 96, 120, and 144 hours after the initiation of transfection. In T24 cells, inhibition of Bcl-X by siRNA was found to be maximal at 24 hours post transfection and had returned to normal levels by day 5. The results were similar for ASO treatment except that maximal activity was achieved at the 8-hour time point. In both cases activity began to taper off after 72 hours and by 120 hours there was no significant inhibition of targeted message with either the ASO or the siRNA.

### Example 11

### Effects of Targeting Intron Sequences

Previous reports have suggested that the site of action of siRNA mediated mRNA degradation in mammalian cells is confined primarily to the cytoplasm (Elbashir et al. (2002), Methods, 26(2), 199-213; Kisielow et al. (2002), Biochem. J., 363,1-5). Conversely, the site of action of RNase H-dependent antisense oligonucleotides has been proposed to be the nucleus of the cell (Condon et al. (1996), Journal of Biological Chemistry, 271(48), 30398-30403; Sazani, P., et al. (2001), Nucleic Acids Research, 29(19), 3965-3974). Since siRNA activity appears limited to the cytoplasm in mammalian cells one would expect that siRNAs targeted to intronic sequences of the pre-mRNA would not reduce target expression. On the other hand, ASOs have been shown to effectively reduce message when targeted to intron sequences (Wickstrom, E. (2001) Mol Biotechnol, 18(1), 35-55).

In order to compare the site of activity of ASOs and siRNAs directly, siRNAs were designed based upon several previously identified active ASO sites that target intron sequences (shown in Table III). The target sites COREST and PAK1 are contained completely within the introns indicated in Table III, while the target sites for caspase recruitment domain 4 and Notch homolog 2 overlap the indicated intron/exon boundary with 10 nucleotides on either side. T24 cells were treated with the ASO or the corresponding siRNA at a single dose of 200 nM as described above. The following day RNA was isolated and message levels for targeted genes ascertained by qRT/PCR. In all cases the ASO effectively reduced the message while an ASO targeted to another gene had no effect on gene expression. In contrast, the homologous siRNAs did not reduce mRNA levels for any of the 5 genes in which introns were targeted nor was any non-specific reduction observed using siRNAs targeted to other genes. As a control one gene, C-raf, was included in which the target was in the exon. As previously demonstrated, the siRNA targeted to the c-raf exon reduced message expression. Although not wishing to be bound by the theory, at least in this instance this data supports the hypothesis that siRNA activity is primarily cytoplasmic.

### Example 12

### Design and Screening of Duplexed Antisense Compounds

In accordance with the present invention, a series of nucleic acid duplexes comprising the antisense compounds of the present invention and their complements can be designed to target desired genes. In some embodiments, the nucleobase sequence of the antisense strand of the duplex comprises at least an 8-nucleobase portion of the nucleotide sequence of the gene of interest. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO:109) and having a two-nucleobase overhang of deoxythymidine(dT) would have the following structure:

| | | |
|---|---|---|
| cgagaggcggacgggaccgTT | Antisense | SEQ ID NO:110 |
| \|\|\|\|\|\|\|\|\|\|\|\|\|\|\|\|\|\|\| | Strand | |
| TTgctctccgcctgccctggc | Complement | SEQ ID NO:111 |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 μM. Once diluted, 30 μL of each strand is combined with 15 μL of a 5X solution of annealing buffer. The final concentration of said buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM. This solution can be stored frozen (−20° C.) and freeze-thawed up to 5 times.

Once prepared, the duplexed antisense compounds are evaluated for their ability to modulate RNA expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN™ Reagent (transfection reagent; Invitrogen, Carlsbad, Calif.) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 13

### Activity of PTEN Antisense Oligoribonucleotides (asRNA)

In Vitro Studies-Primary Hepatocytes

The antisense oligoribonucleotides of the present invention were used to treat mouse primary hepatocytes, and the in vitro activity of these oligomeric compounds was characterized. Mouse primary hepatocytes were dosed at concentrations ranging from 12.5 to 200 nM antisense oligoribonucleotide, and PTEN target mRNA levels were compared to levels in untreated control cells. As shown in Table IV, ISIS 303912, (SEQ ID NO: 112) UUUGUCUCUGGUCCUUACUU, was found to exhibit a dose responsive inhibition of PTEN mRNA levels.

TABLE IV

| | |
|---|---|
| | Percent Inhibition |
| Isis Number | of PTEN mRNA by Dose |

| (SEQ ID NO) | 12.5 nM | 50 nM | 100 nM | 200 nM |
|---|---|---|---|---|
| Control | 0 | 0 | 0 | 0 |
| 303912 (112) | 6 | 35 | 54 | 70 |

In Vitro Studies

Chemical Modifications

The antisense oligoribonucleotides of the present invention were used to treat T24 cells, and the in vitro activity of these oligomeric compounds was characterized. T24 cells were dosed at concentrations ranging from 50 to 200 nM antisense oligoribonucleotide (asRNA), and PTEN target mRNA levels were compared to levels in untreated control cells. Chemical modifications were made to ISIS 303912 and compared to the parent compound for their ability to reduce mRNA levels in T24 cells. ISIS 316449 (which represents ISIS 303912 with three 2′-O-methoxyethyl (2′-O methyl) modifications on the 3′ end and a 5′ terminal phosphate) and ISIS 319022 (which represents ISIS 303912 having fully modified 2′-F modifications throughout and a 5′ terminal phosphate) were compared to ISIS 303912 also having a 5′ terminal phosphate. As shown in Table V, ISIS 303912, (SEQ ID NO: 112) UUUGUCUCUGGUCCUUACUU, as well as ISIS 316449 and ISIS 319022 all were found to exhibit dose responsive inhibition of PTEN mRNA levels.

The duration of action of ISIS 303912 and ISIS 316449 was also investigated out to 72 hours, with timepoints of 24, 32, 48 and 72 hours. Both compounds maintained at least a 70% target reduction throughout the timecourse.

TABLE V

| | Percent Inhibition | | |
|---|---|---|---|
| Isis Number | of PTEN mRNA by Dose | | |
| (SEQ ID NO) | 50 nM | 100 nM | 200 nM |
| Control | 0 | 0 | 0 |
| 303912 (112) | 35 | 54 | 70 |
| 316449 | 40 | 70 | 75 |
| 319022 | 20 | 55 | 50 |

Mismatches in Double Stranded Constructs

Additionally, the activity of double-stranded oligoribonucleotide compounds of the present invention representing PTEN chimeric RNA constructs bearing seven 2′-O—methyl substitutions at the 3′-terminus of either the sense strand, the antisense strand or both strands was also compared in vitro in T24 cells. When the seven 2′-O-methyl substitutions were at the 3′-end of both strands, target levels were reduced by 75%. When the seven 2′-O-methyl substitutions were at the 3′-end of the sense strand, target levels were reduced by 70%; and when the seven 2′-O methyl substitutions were at the 3′-end of the antisense strand, target levels were reduced by 80%.

In Vivo Studies

To characterize the in vivo activity of the oligomeric compounds of the present invention, Balb/c mice were treated with compounds of the invention and levels of target were measured in several tissues. The oligomeric compounds of the study and their sequence are shown in Table VI. ISIS 116847, (SEQ ID NO: 113), represents an antisense oligodeoxyribonucleotide. ISIS 22023, (SEQ ID NO: 114) represents an off-target oligodeoxyribonucleotide used as a negative control. All oligonucleotides are full phosphorothioate, bold letters indicate 2′-O-methoxyethyl substitutions, and bold italicized letters indicate 2′-O-methyl substitutions.

TABLE VI

| ISIS | | | |
|---|---|---|---|
| Number | Sequence | Target Gene | SEQ ID NO: |
| 116847 | CTGCTAGCCTCTGGATTTGA | PTEN | 113 |
| 22023 | TCCAGCACTTTCTTTTCCGG | Fatty acid synthase | 114 |
| 316449 | UUUGUCUCUGGUCCUUACUU | PTEN | 115 |
| 335435 | UUUAUCGCUUCUCGUUGCUU | Mismatch control | 116 |

For each oligomeric compound studied, male mice from the inbred Balb/c strain (Charles River, Wilmington, Mass.), weighing about 20 g, were used. Following a 1-week acclimatization, the animals received a single subcutaneous injection of the compound (200 μL; 50 mg/kg) followed by three tail vein injections (200 μL; 5 mg/kg) for a total of four injections. Each injection was administered every other day and the compounds were administered in phosphate buffered saline (PBS), pH 7.0.

Control groups consisted of animals injected with saline (saline+10.5% PBS) or a control mismatch compound. All control animals were treated in the same manner as experimental animals. The control mismatch compound was injected at the same dose as the oligomeric compound of the invention.

At the end of the treatment period, the mice were sacrificed and tissues were collected for immediate evaluation. Tissues can be frozen on dry ice and stored at −80° C. for future analysis. The tissues collected included liver, kidney, lung, spleen and heart and these were evaluated for target mRNA expression level by quantitative real-time PCR, as described in other examples herein. Protein levels can also be evaluated by immunoblot analysis. Serum can also be collected, for the purpose of analyzing cholesterol, triglycerides, free fatty acids, glucose, insulin and liver enzymes.

The tissues may also be prepared for routine histological analysis, which allows the assessment of nuclear and cellular structure and appearance, as well as the visualization of specific proteins by direct or indirect immunofluorescence. The expression of genes that interact with the target gene product, either indirectly or in the same pathway, can also be evaluated by real-time PCR, using primers and probes designed to the mRNA of interest, and immunoblot or immunohistochemical analysis using antibodies that specifically recognize the proteins of interest.

The weight, food consumption and metabolic rate of each mouse can also be analyzed. Blood can be obtained via retro-orbital collection during the study, or at the termination of the study by cardiac puncture. One retro-orbital bleed (either 0.25, 0.5, 2 or 4 lv post dose) and a terminal bleed (either 1, 3, 8 or 24 h post dose) is collected from each group. The terminal bleed (approximately 0.6-0.8 ml) is collected by cardiac puncture following ketamine/xylazine anesthesia. The blood is transferred to an EDTA-coated collection tube and centrifuged to obtain plasma.

The in vivo activity of the chimeric oligomeric constructs in liver tissue is shown in Table VII.

TABLE VII

| ISIS Number | | |
|---|---|---|
| (SEQ ID NO) | Target Gene | Percent inhibition of PTEN mRNA |
| saline | | 0 |
| 116847 (113) | PTEN | 87.2 |
| 22023 (114) | Fatty acid synthase | 10.1 |
| 316449 (115) | PTEN | 37.6 |
| 335435 (116) | Mismatch control | 23.8 |

The antisense oligodeoxyribonucleotide, ISIS 116847 (SEQ ID NO: 113), showed significant inhibition of target. Furthermore, the antisense oligoribonucleotide, ISIS 316449 (SEQ ID NO: 115), bearing three 2′-O methyl substitutions at the 3′-end exhibited a greater effect on target reduction than did the mismatch control oligo, ISIS 335435 (SEQ ID NO: 116) bearing the same three 2′-O methyl substitutions at the 3′-end.

### Example 14

### Active siRNAs Tolerate Multiple and Periodic Mismatches

In addition to the MM2_{—}1 double-stranded siRNA construct bearing 2 base mismatches at the ends, and the MM2_{—}2 double-stranded siRNA bearing two base mismatches in the center shown in Table VIII, a third double-stranded oligodeoxyribonucleotide siRNA construct, MM6, targeting PTEN but bearing six mismatched basepairs incorporated throughout was designed and tested for its effect on PTEN mRNA levels. Bases mismatched against the PTEN mRNA are shown in bold.

TABLE VIII

| | | |
|---|---|---|
| duplex: (SEQ ID | | |
| NO of top | | |
| strand/bottom | | Percent Inhibition |
| strand (5′-3′)) | Sequence | of PTEN mRNA |

| | | |
|---|---|---|
| MM2-1 | CCAAUCCAGAGGCUAGAAGdTdT | 45 |
| (117/118) | dTdTGGUUAGGUCUCCGAUCUUC | |
| MM2-2 | CAAAUCCGGAAGCUAGCAGdTdT | 1 |
| (118/119) | dTdTGUUUAGGCCUUCGAUCGUC | |
| MM6 | CUAAACCGGAUGCCAGAAGdTdT | 15 |
| (120/121) | dTdTGAUUUGGCCUACGGUCUUC | |

The MM6 double-stranded oligoribonucleotide construct with imperfect sequence specificity for the PTEN mRNA retains some ability to act as a siRNA; perfect Watson-Crick base pairing does not appear essential for siRNA activity.

Accordingly, in some situations in designing oligomeric compound, the oligomeric compound may be designed by balancing several factors, including, but not limited to, activity of the oligomeric compound, nuclease stability, efficiency of delivery, ease of manufacturing, among others. For example, in some scenarios it may be desired to sacrifice some activity of the oligomeric compound in order to improve delivery of the oligomeric compound to its target.

We claim:

1. A method of identifying a multifunctional oligomeric compound to modulate expression of RNA comprising:
(a) contacting a target RNA with one or more double-stranded oligomeric compounds hybridizable to one or more target regions of said RNA and identifying double-stranded oligomeric compounds which inhibit target RNA levels by at least 50%;
(b) contacting the target RNA with an antisense strand of said modulating double-stranded oligomeric compound and determining whether the antisense strand inhibits target RNA levels by at least 50%; and
(c) identifying said inhibiting antisense strand and said inhibiting double-stranded oligomeric compound as multifunctional oligomeric compounds.

2. A multifunctional oligomeric compound identified according to claim 1.

3. A method of claim 1 wherein the multifunctional oligomeric compound inhibits target RNA levels by at least 80%.

4. The method of claim 1 wherein the target region is identified by a single-stranded oligomeric gene walk across the target RNA.

5. The method of claim 1 wherein the target region is identified by secondary structure analysis of the target RNA.

6. The method of claim 1 wherein said target region is at least a portion of an induced gene.

7. The method of claim 6 wherein the induced gene is CD54.

8. The method of claim 1 wherein said target region is at least a portion of a constitutive gene.

9. The method of claim 1 wherein said target region is localized to the 3′UTR, the 5′UTR, an intron:exon boundary, an exon:exon boundary, a start region or a coding region of the RNA.

10. The method of claim 1 wherein said target region is localized to the 3′UTR.

11. The method of claim 1 wherein said target region is localized to the 5′UTR.

12. The method of claim 1 wherein said target region is localized to an intronic portion of a gene.

13. The method of claim 1 wherein said target region is localized to an exon.

14. The method of claim 1 wherein said target region is localized to an intron/exon boundary.

15. The method of claim 1 wherein said target regions overlaps the intron/exon boundary with 5-10 nucleotides on either side of the boundary.

16. A method for optimizing target region selection for modulation of RNA expression comprising:
(a) contacting one or more double-stranded oligomeric compounds with one or more regions of a target RNA and identifying target regions which, when contacted with the one or more double-stranded oligomeric compounds, result in inhibition of target RNA levels of at least 50%;
(b) contacting one or more single-stranded oligomeric compounds with said inhibited target regions and identifying regions which, when contacted with the one or more single-stranded oligomeric compounds, result in inhibition of target RNA levels of at least 50%;
(c) identifying regions modulated by at least one double-stranded oligomeric compound and at least one single-stranded oligomeric compound as optimized target regions.

17. The method of claim 16 wherein target RNA levels are inhibited by at least 80% by single-stranded oligomeric compounds and double-stranded oligomeric compounds.

18. The method of claim 1 wherein the oligomeric compound is an antisense oligonucleotide.

19. The method of claim 1 wherein the oligomeric compound has at least one modification of the base, sugar or internucleoside linkage.

20. The method of claim 1 wherein the oligomeric compound has a modification at the 2′ position of at least one sugar.

21. The method of claim 1 wherein said oligomeric compound comprises at least four consecutive 2′-hydroxyl ribonucleosides and at least one modified nucleoside.

22. The method of claim 1 wherein said oligomeric compound is from about 12 to about 50 nucleotides in length.

23. The method of claim 1 wherein said oligomeric compound is from about 18 to about 25 nucleotides in length.

24. The method of claim 1 wherein said oligomeric compound comprises at least four consecutive 2′-hydroxyl ribonucleosides and at least one modified nucleoside; said modified nucleoside adapted to modulate at least one of; binding affinity or binding specificity of said oligomeric compound.

25. The method of claim 1 wherein the oligomeric compound is RNA.

26. The method of claim 1 wherein the oligomeric compound is a siRNA

27. The method of claim 1 wherein said hybridization is under moderate or high stringency conditions.

28. The method of claim 1 wherein the oligomeric compound is a potent modulator of the target RNA.

29. The method of claim 1 wherein the oligomeric compound is a gapmer.

30. The method of claim 1 wherein the oligomeric compound comprises at least six consecutive nucleosides with 2′ modifications.

31. The method of claim 1 wherein the oligomeric compound is a hemimer.

32. The method of claim 1 wherein the oligomeric compound comprises at least one phosphorothioate linkage.

33. The method of claim 1 wherein the oligomeric compound is a chimeric compound.

34. The method of claim 1 wherein the oligomeric compound comprises one or more chimeric regions.

35. The method of claim 1 wherein the target RNA is preselected.

36. A method of modulating RNA expression comprising contacting target regions optimized according to claim 16 with two or more oligomeric compounds.

37. A method of optimizing modulation of RNA comprising contacting a target RNA with at least two oligomeric compounds hybridizable to a target region of said target RNA wherein at least two oligomeric compounds each inhibit RNA levels by at least 50% when tested individually.

38. A method of optimizing target regions of RNA comprising:
(a) contacting a target RNA comprising a target region with a plurality of oligomeric compounds hybridizable with said target region; and,
(b) identifying target regions as optimized when two or more of said oligomeric compounds inhibit target RNA levels by at least 50%.

39. The method of claim 38 wherein the oligomeric compound comprises at least one double-stranded region.

40. The method of claim 38 wherein target regions are identified as optimized when two or more of said oligomeric compounds inhibit target RNA levels by at least 80%.

41. A method of selecting a target region of a gene comprising:
(a) contacting a target RNA comprising at least one target region with a plurality of oligomeric compounds hybridizable with said at least one target region, wherein said oligomeric compounds comprise at least one siRNA oligomeric compound and at least one ASO oligomeric compound;
(b) identifying siRNA and ASO oligomeric compounds which inhibit RNA levels by at least 60% for each of said at least one target regions; and
(c) selecting target regions when there is a significant association between inhibiting siRNA oligomeric compounds and ASO oligomeric compounds for the target region.

42. The method of claim 41 wherein at least one of said oligomeric compounds comprises at least one double-stranded region.

43. A method of claim 41 wherein (c) is performed using a ROC analysis.

44. A method of claim 43 wherein the ROC analysis yields an area under the curve of at least 0.6.

45. A method of claim 43 wherein the ROC analysis yields an area under the curve of at least 0.8.

46. A target region of a gene selected according to the method of claim 41.

47. A method of selecting an optimized single-stranded oligomeric compound comprising:
(a) contacting a target RNA with one or more double-stranded oligomeric compounds;
(b) identifying one or more double-stranded oligomeric compounds which inhibit target RNA levels by at least 50%; and
(c) selecting the strand of the double-stranded oligomeric compound that hybridizes to the target RNA as the optimized single-stranded oligomeric compound.

48. The method of claim 47 wherein target RNA levels are inhibited by at least 80%.

49. A method of selecting an optimized double-stranded oligomeric compound comprising:
(a) contacting a target RNA with one or more single-stranded oligomeric compounds;
(b) identifying one or more single-stranded oligomeric compounds which inhibit target RNA levels by at least 50%; and
(c) hybridizing a complementary single-stranded oligomeric compound to said single-stranded oligomeric compound, thereby yielding an optimized double-stranded oligomeric compound.

50. A method of selecting a single-stranded oligomeric compound comprising;
(a) contacting a target RNA with one or more double-stranded oligomeric compounds;
(b) identifying one or more double-stranded oligomeric compounds which inhibit target RNA levels by at least 50%; and
(c) selecting the strand of the identified double-stranded oligomeric compound which is complementary to the target RNA as the selected single-stranded oligomeric compound.

51. A method of selecting a double-stranded oligomeric compound comprising:
(a) contacting a target RNA with one or more single-stranded oligomeric compounds;
(b) identifying one or more single-stranded oligomeric compounds which inhibit target RNA levels by at least 50%; and
(c) hybridizing a complementary single-stranded oligomeric compound to said identified single-stranded oligomeric compound, yielding a double-stranded oligomeric compound as the selected double-stranded oligomeric compound.

52. A method of identifying one or more optimized double-stranded oligomeric compounds comprising:
(a) cloning one or more target regions from a target RNA into a vector/plasmid construct;
(b) transfecting said vector/plasmid into a cell;
(c) contacting a cell transfected with said vector/plasmid with one or more double-stranded oligomeric compounds, said compounds having one strand hybridizable to said target region; and,
(d) identifying one or more double-stranded oligomeric compounds which inhibit target RNA levels by at least 50%.

53. An oligomeric compound, 8-80 nucleobases in length, targeted to a target RNA, wherein said oligomeric compound specifically hybridizes said target RNA and wherein said oligomeric compound inhibits RNA levels by at least 50% in both single-stranded and double-stranded forms.

54. The oligomeric compound of claim 53 wherein the oligomeric compound comprises one or more hairpin regions.

55. The oligomeric compound of claim 53 wherein RNA levels are measured in A549 cells.

56. An oligomeric compound, 8-80 nucleobases in length targeted to a target RNA, wherein said oligomeric compound has a least 80% sequence homology to the complement of said target RNA and wherein said oligomeric compound inhibits RNA levels by at least 60% in both single-stranded and double-stranded forms.

57. The oligomeric compound of claim 56 wherein the sequence homology is at least 90%.

58. The oligomeric compound of claim 56 wherein the oligomeric compound has at least 2 mismatches as compared to the complement of the target RNA.

59. The oligomeric compound of claim 58 wherein the mismatches are internal or external base mismatches.

60. The oligomeric compound of claim 56 wherein no more than two of the four 3′-most nucleotides of the oligomeric compound are mismatches.

61. The oligomeric compound of claim 56 wherein said oligomeric compound has an IC₅₀ no greater than 100 nM.

62. The oligomeric compound of claim 56 wherein said oligomeric compound has an IC₅₀ no greater than 10 nM.

63. The oligomeric compound of claim 56 wherein said oligomeric compound is targeted to the 3′UTR, the 5′UTR, an intron:exon boundary, an exon:exon boundary, a start region or a coding region of the RNA.

64. The oligomeric compound of claim 56 wherein said oligomeric compound is targeted to the 3′UTR.

65. The oligomeric compound of claim 56 wherein said oligomeric compound is targeted to the 5′UTR.

66. The oligomeric compound of claim 56 wherein said oligomeric compound is targeted to an intronic portion of the RNA.

67. The oligomeric compound of claim 56 wherein said oligomeric compound is targeted to an exon.

68. The oligomeric compound of claim 56 wherein said oligomeric compound is targeted to an intron/exon boundary.

69. The oligomeric compound of claim 56 wherein said oligomeric compound has alternating linkages.

70. The oligomeric compound of claim 56 wherein the oligomeric compound has alternating modifications.

71. The oligomeric compound of claim 56 wherein every second nucleotide in the antisense strand of the double stranded oligomeric compound is modified.

72. The oligomeric compound of claim 71 wherein the first modified nucleotide is the 5′-most nucleotide of the oligomeric compound.

73. The oligomeric compound of claim 71 wherein the modifications are 2′ modifications.

74. The oligomeric compound of claim 71 wherein the modifications are one or more of 2′-O alkyl, 2′-O-methoxyethyl, 2′-methoxyethoxy, 2′-dimethylaminooxyethoxy, 2′-dimethylaminoethoxyethoxy, 2′-methoxy, 2′-aminopropoxy, 2′-allyl, 2′-O-allyl (2′-O—CH₂—CH═CH₂), or 2′-fluoro.

75. The oligomeric compound of claim 56 wherein said oligomeric compound comprises:
a first segment;
a second segment; and,
a third segment comprising three or four nucleobases, said third portion located between said first and second segments;
wherein said first and second segments each have at least one modified nucleobase.

76. The oligomeric compound of claim 75 wherein said third segment has no modified nucleobases.

77. The oligomeric compound of claim 75 wherein said first and second segments each comprise at least one modified linkage/modification.

78. The oligomeric compound of claim 77 wherein said third segment has no modified linkages or modifications.

79. The oligomeric compound of claim 56 wherein said oligomeric compound hybridizes to at least a portion of the 3′ UTR of said target RNA.

80. The oligomeric compound of claim 56 wherein said oligomeric compound comprises at least four consecutive 2′-hydroxyl ribonucleosides and at least one modified nucleoside; said modified nucleoside adapted to modulate at least one of; binding affinity or binding specificity of said oligomeric compound.

81. The oligomeric compound of claim 56 wherein said oligomeric compound comprises at least seven 2′-O-methyl substitutions at the 3′-terminus of the oligomeric compound.

82. An oligomeric compound of claim 53 wherein the oligomeric compound has at least six mismatches as compared to the complement of the target RNA.
